(19) United States
(12) Patent Application Publication
(10) Pub. No.: US 2023/0167449 A1
(43) Pub. Date: Jun. 1, 2023

(54) DEPLETION OF EXT1 EXPRESSION AND/OR ACTIVITY IMPROVES CELLULAR PRODUCTION OF BIOLOGICAL ENTITIES

(71) Applicant: UNIVERSITÉ DE LIÈGE, Liège (BE)

(72) Inventors: Jean-Claude TWIZERE, Liège (BE); Despoina KERSELIDOU, Liège (BE)

(73) Assignee: UNIVERSITÉ DE LIÈGE, Liège (BE)

(21) Appl. No.: 17/904,641

(22) PCT Filed: Feb. 19, 2021
(86) PCT No.: PCT/EP2021/054190

§ 371 Date: Aug. 19, 2022

(30) Foreign Application Priority Data
Feb. 21, 2020 (EP) 20158875.3

(51) Int. Cl. C12N 15/113; C12N 7/00; C07K 14/705

(52) U.S. Cl. CPC C12N 15/1137; C07K 14/705; C12N 7/00; C12Y 204/01224; C12Y 204/01225; C12N 2310/14; C12N 2310/531; C12N 2740/15052; C12N 2750/14152

(57) ABSTRACT

The use of an inhibitor of EXT1 expression and/or activity for the production of a biological entity in a cell. Also, the use of a cell having at least depleted EXT1 expression and/or activity for the production of a biological entity. Further, evidence is provided about the role of glycosylation in rapid dynamism of ER shaping and function. In particular, depletion of EXT1 results in a recomposed ER shaping, which could benefit production of recombinant proteins.

### FIELD OF INVENTION

The present invention relates to eukaryotic cells as host system for the production of biological entities, such as recombinant polypeptides (or proteins) or viral particles. More particularly, the invention relates to the depletion of EXT1 for improving recombinant polypeptide or viral particles in eukaryote host cells.

### BACKGROUND OF INVENTION

The market for recombinant technology, including recombinant proteins, recombinant monoclonal antibodies and recombinant vaccines has increased in the past decade. However, insufficient production capacities have become the limitation step for the development of recombinant drugs. Production gap also results in expensive prices.

The production of recombinant biological entities, such as, e.g., recombinant proteins, viral particles, as vectors for therapy, etc, suffers from several bottlenecks, such as the choice of the producing host (bacteria vs mammalian cells), the culture of said host allowing quantitative and qualitative amounts of said recombinant entity (serum-free and/or feeder-free culture).

Mammalian cells and bacterium E. coli are currently one of the most important production hosts for recombinant proteins. E. coli is noticeably used for the production of recombinant proteins of therapeutic value that do not require post-translational modifications, such as, e.g., insulin, growth hormone, beta interferon, and interleukins.

Moreover, for human and veterinary therapy, production of recombinant proteins of therapeutic value in mammalian cell host is often necessary to achieve adequate post-translational modifications, such as, e.g., the correct folding of proteins (including disulfide bridges formation), the correct glycosylation, the correct phosphorylation. Appropriate folding and assembly would depend on the correct handling of the yet to be synthesized recombinant entity, in particular by two specialized organelles within the cells: the endoplasmic reticulum (ER) and the Golgi apparatus. In addition, viral-based gene therapy is a rapidly growing field. However, production titer is a key factor, since high titer results in a smaller, more cost-effective, production process. Higher titer production of viral vectors can lower reagent demand labor, and facility requirements.

The ER is the largest organelle in the eukaryotic cell, spanning from the nuclear envelope to the plasma membrane and establishing functional communication channels with other organelles that in turn, influence its physical properties and functions (Phillips and Voeltz, 2016). During normal cell homeostasis, the complex network of ER tubules and flat matrices is in a continuous motion to support the synthesis and distribution of proteins and lipids traversing the ER luminal space (Palade and Porter, 1954). The basic structure of high-curvature regions such as tubules and edges of ER sheets is built by the oligomerization of proteins with hydrophobic hairpin domains, reticulons (RTNs), and receptor expression enhancing proteins (REEPs) (Voeltz et al., 2006). ER membranes are fused, in a homotypic manner, by atlastin (ATL) GTPases that dimerize in opposing layers (Liu et al., 2015). In mammalian cells, curvature structures of sheet membranes are stabilized by a luminal bridging protein, the cytoskeleton-linking membrane protein 63 (CLIMP63) (Shibata et al., 2010). ER sheets are then stacked as interconnected helicoidal motifs that form a continuous three-dimensional network resembling a parking garage (Terasaki et al., 2013).

It is currently believed that cooperation between ER shape and luminal dynamics dictates ER functions (Schwarz and Blower, 2016). While ER sheets are the primary sites for translation, translocation and folding of integral membranes and secreted proteins, ER tubules are thought to be more involved in other ER functions such as lipid synthesis and interactions with other organelles (Voeltz et al., 2002; Shibata et al., 2006). Cells actively adapt their ER tubules/sheets balance and dynamics to coordinate ER morphology and function, in accordance with cellular demands (Westrate et al., 2015). However, the molecular mechanisms underlying this overall maintenance and flexibility of the ER network remain obscure. While permanent interactions between membrane curvature proteins are sufficient to form the basic ER structure, transient protein-protein interactions such as translational modifications (TMs) may participate in the dynamic shaping of the mammalian ER. Among TMs, glycosylation is a conserved post- or co-translational modification involved in many cellular processes including cell-fate determination and biological diversity. Synthesis of glycans and attachment to the acceptor peptide initiates in the ER and terminates in the Golgi apparatus by multi-step sequential activities of glycosyltransferases and glycosidases, competing for activated glycans and overlapping substrates (Reily et al., 2019). The final composition of oligosaccharide chains bound to a glycoprotein depends not only on enzymes expression and localization but also on the availability and heterogeneity of sugar substrates.

Glycosylation is well known to regulate the physical properties of different glycolipid and glycoprotein biopolymers at the surface of mammalian cells by controlling plasma membrane and cell coat morphologies (Shurer et al., 2019). The impact of glycosylation of ER membrane components and the quantitative and qualitative contributions of glycan structures potentially attached to the ER membrane and resident proteins are entirely unknown.

There is a need to provide the state of the art with eukaryotic host cell systems with improved capacity for producing recombinant proteins or viral particles, as viral vectors.

There is a need to provide the state of the art with universal eukaryotic host cell systems that can be easily generated and handled, for qualitatively producing large amounts of recombinant proteins or viral particles.

### SUMMARY

One aspect of the invention relates to the use of an inhibitor of EXT1 expression and/or activity for the production of a biological entity in a cell.

In some embodiments, said inhibitor of the EXT1 expression and/or activity is selected from a group comprising an oligonucleotide, an aptamer, an oligopeptide, a polypeptide, a chemical compound and an analog thereof. In certain embodiments, said inhibitor of EXT1 expression is an oligonucleotide having at least 75% identity with any one of sequences SEQ ID NO: 1 to SEQ ID NO: 27 and SEQ ID NO: 33 to SEQ ID NO: 53. In some embodiments, said inhibitor of the EXT1 expression is an oligonucleotide represented by any one of sequences SEQ ID NO: 1 to SEQ ID NO: 27 and SEQ ID NO: 33 to SEQ ID NO: 53. In certain embodiments, the biological entity is selected in a group comprising a recombinant polypeptide and/or a viral particle. In some embodiments, the cell is a eukaryote cell.

Another aspect of the invention also pertains to the use of a cell having at least depleted EXT1 expression and/or activity for the production of a biological entity. In some embodiments, the cell is a eukaryotic cell. In certain embodiments, the biological entity is selected in a group comprising a recombinant polypeptide and/or a viral particle. In some embodiments, the cell comprises a partial or total knockout of the EXT1 gene. In certain embodiments, the at least depleted EXT1 expression and/or activity is obtained by the treatment of said cell with an inhibitor of EXT1 expression and/or activity. In some embodiments, said inhibitor of the EXT1 expression and/or activity is selected from a group comprising an oligonucleotide, an aptamer, an oligopeptide, a polypeptide, a chemical compound and an analog thereof. In certain embodiments, said inhibitor of the EXT1 expression is selected in a group comprising an oligonucleotide having at least 75% identity with any one of sequences SEQ ID NO: 1 to SEQ ID NO: 27 and SEQ ID NO: 33 to SEQ ID NO: 53, preferably is an oligonucleotide represented by any one of sequences SEQ ID NO: 1 to SEQ ID NO: 27 and SEQ ID NO: 33 to SEQ ID NO: 53.

In a still other aspect, the invention relates to a method for the production of a biological entity in a cell, said method comprising the steps of:
a) providing a cell population having at least depleted EXT1 expression and/or activity;
b) transfecting the cell population of step a) with an oligonucleotide encoding the biological entity, preferably a polypeptide or a viral particle.

Another aspect of the invention relates to a method for the production of a biological entity in a cell, said method comprising the steps of:
a) providing a cell population;
b) transfecting the cell population of step a) with an oligonucleotide encoding the biological entity, preferably a polypeptide or a viral particle.
c) inhibiting EXT1 expression and/or activity in the said cell by using an EXT1 inhibitor as defined herein.

### DEFINITIONS

In the present invention, the following terms have the following meanings:
“About”, preceding a figure, means plus or less 10% of the value of said figure.
“Comprises” is intended to mean “contains”, “encompasses” and “includes”. In some embodiments, the term “comprises” also encompasses the term “consists of”.
“EXT1” refers to the Exostosin Glycosyltransferase 1. The gene EXT1 or the protein EXT1 may also be refer to as the Glucuronosyl-N-Acetylglucosaminyl-Proteoglycan/N-Acetylglucosaminyl-Proteoglycan 4-Alpha-N-Acetylglucosaminyl-transferase, Glucuronosyl-N-Acetylglucosaminyl-Proteoglycan 4-Alpha-N- Acetylglucosaminyl-transferase, N-Acetylglucosaminyl-Proteoglycan 4-Beta-Glucuronosyl-transferase, Langer-Giedion Syndrome Chromosome Region, Putative Tumor Suppressor Protein EXT1, Multiple Exostoses Protein 1, Exostosin-1, enzyme EC 2.4.1.224 or EC 2.4.1.225, TRPS2, LGCR, EXT, TTV, or LGS.
“Expression” refers to the transcription and/or translation of a particular nucleotide sequence driven by a promoter. By extension, “EXT1 expression” is intended to refer to the synthesis of the EXT1 mRNA or the EXT1 polypeptide within a cell.
“Activity” refers to the biological function of a polypeptide. By extension, “EXT1 activity” is intended to refer to the enzymatic function of the EXT1 polypeptide, i.e., the glycosyl-transferase activity, that can be measured in vivo or in vitro.
“Inhibitor” refers to a natural or synthetic compound that has the biological effect of inhibiting, significantly reducing, or down-regulating the expression of a gene and/or a polypeptide or that has the biological effect inhibiting, significantly reducing, or down-regulating the biological activity of a polypeptide, as compared to physiological expression or activity levels. By extension, an “EXT1 inhibitor” refers to a compound that has the biological effect of inhibiting or significantly reducing or down-regulating the expression of the gene encoding the EXT1 polypeptide and/or the expression of the EXT1 polypeptide and/or the biological activity of the EXT1 polypeptide.
“Biological entity” refers to an organic product that can be produced naturally or artificially (e.g., by recombinant technologies). Peptides, polypeptides, proteins, viral vectors and viral particles are non-limitative examples of biological entities.
“Recombinant peptide, polypeptide or protein” refers to a peptide, polypeptide or protein generated from recombinant DNA, i.e., from DNA artificially inserted in a producing host cell.
“Viral particle” refers to a particle of viral origin that consists of a nucleic acid core (either RNA or DNA) surrounded by a polypeptide coat, optionally with external envelopes and that is the extracellular infectious form of a virus.
“Knockout” refers to a genetic mutation resulting in a loss of function and/or a loss of expression of the polypeptide encoded by the said gene. In one embodiment, said genetic mutation corresponds to the disruption of all or a portion of a gene of interest, preferably the total disruption of the gene. Preferably, the deletion starts at or before the start codon of the deleted gene, and ends at or after the stop codon of the deleted gene. Other examples of genetic mutations include, but are not limited to, substitution, deletion, or insertion.
“Depletion” refers to a partial or total reduction of the expression and/or activity of a polypeptide. By extension, “depleted EXT1 expression and/or activity” is intended to relate to a significant reduction in the expression and/or activity of EXT1 polypeptide.
“Transfection” refers to a process by which exogenous nucleic acid is transferred or introduced into a host cell, in particular a host cell of eukaryotic origin. A “transfected” host cell is one which has been manipulated so as to incorporate the exogenous nucleic acid. The cell includes the primary subject cell and its progeny.

### DETAILED DESCRIPTION

The inventors provide herein evidences about the role of glycosylation in rapid dynamism of ER shaping and function. Super-resolution imaging has allowed to show that, Exostosin-1 (EXT1), an ER-resident glycosyltransferase known to polymerize heparan sulfate (HS) chains by sequential addition of glucuronic acid and N-acetylglucosamine molecules, is localized in dense sheets and ER tubules. Then, using specific ER dynamic and trafficking reporters, RNA sequencing (RNA-seq), quantitative proteomics, and glycomics analyses, the morphology and molecular composition of ER membranes in cells depleted for EXT1, were systematically explored. The inventors unexpectedly uncovered a relationship between ER extension and reprogramming of glycan molecules linked to ER membrane proteins. Thus, glycosylation provides an additional layer of regulation contributing to the heterogeneity of ER morphologies in response to different cell types and states. In addition, the inventors have observed that EXT1 depletion results in a reshaping of the morphology of both the ER and the Golgi apparatus. These observations provide a basis for using EXT1-depleted cells for improving the production of recombinant proteins or recombinant viral particles, as illustrated by the examples below.

State of the art previously disclosed silencing of EXT1. For example, document EP3604502A1 disclosed that silencing EXT1 by CRISPR-Cas9 technique resulted in an absence of heparan sulfate, so as to stabilize the production of enterovirus 71 in order to screen vaccines against hand, foot and mouth disease. In addition, CN107058476A disclosed that silencing EXT1, by a shRNA, may be useful to treat liver cancer.

Here, in some aspect, the invention relates to a use of an inhibitor of EXT1 expression and/or activity for the production of a biological entity in a cell.

The inventors observed that the metrics of both the ER and the Golgi apparatus, including the length and the perimeter of the cisternae, and the number of cisternae per stack, are significantly altered in cells depleted of EXT1 by an shRNA, as compared to cells with physiological expression level of EXT1. In addition, the inventors observed that production of recombinant proteins or viral particles are significantly increased in said EXT1-depleted cells as compared to control cells. Without wishing to be bound to a theory, the inventors believe that the change of morphology within the ER, the Golgi apparatus and the cell size favorize a larger qualitative and quantitative production of recombinant proteins and/or viral particles. In other words, the invention described herein is intended to provide means to increase the yield of production of recombinant proteins and/or viral particles.

In some embodiments, said inhibitor of the EXT1 expression and/or activity is selected from a group comprising an oligonucleotide, an aptamer, an oligopeptide, a polypeptide, a chemical compound and an analog thereof.

In some embodiments, said oligonucleotide is selected in a group comprising an antisense RNA, a miRNA, a guide RNA, a siRNA, a shRNA.

In certain embodiments, said inhibitor of the EXT1 expression is selected from a siRNA or an shRNA. In some embodiments, said inhibitor of the EXT1 expression is a siRNA. In some embodiments, said inhibitor of the EXT1 expression is an shRNA.

As used herein, “antisense RNA” (also referred to as “asRNA”) refers to a single stranded RNA that is complementary to a protein coding messenger RNA (mRNA) with which it hybridizes, and thereby blocks its translation into protein.

As used herein, “miRNA” (also referred to as “miR”) refers to a non-coding RNA of about 18 to about 25 nucleotides in length. These miRNAs could originate from multiple origins including: an individual gene encoding for a miRNA, from introns of protein coding gene, or from poly-cistronic transcript that often encode multiple, closely related miRNAs. In the following disclosure, the standard nomenclature system is applied, in which uncapitalized “mir-X” refers to the pre-miRNA (precursor), and capitalized “miR-X” refers to the mature form. When two mature miRNAs originate from opposite arms of the same pre-miRNA, they are denoted with a -3p or -5p suffix. In the following disclosure, unless otherwise specified, the use of the expression miR-X refers to the mature miRNA including both forms -3p and -5p, if any. Within the scope of the invention, the expressions microRNA, miRNA and miR designate the same compound.

As used herein, “guide RNA” (also referred to as “gRNA” or “sg RNA”) refers to a non-coding short RNA sequence that binds to the complementary target DNA sequence. In practice, the guide RNA may be used for DNA editing involving CRISPR-Cas system.

As used herein, “siRNA” (also referred to as “silencer RNA”, “silencing RNA”, “short interfering RNA” or “small interfering RNA”) refers to double-stranded RNA (dsRNA), having a length generally comprised from about 20 bp to 25 bp, having phosphorylated 5′ ends and hydroxylated 3′ ends with two overhanging nucleotides. siRNAs interfere with the expression of specific genes with complementary nucleotide sequences by degrading mRNA after transcription, thereby preventing translation.

As used herein, “shRNA” (also referred to as “short hairpin RNA” or “small hairpin RNA”) refers to an artificial RNA having a tight hairpin turn that can be used to silence target gene expression.

As used herein, “aptamer” refers to a nucleic acid that binds to a specific target molecule.

In some embodiment, the inhibitor of EXT1 expression is an oligonucleotide having at least 75% identity with any one of sequences SEQ ID NO: 1 to SEQ ID NO: 27 and SEQ ID NO: 33 to SEQ ID NO: 53. In certain embodiments, the inhibitor of EXT1 expression is an oligonucleotide having at least 75% identity with any one of sequences SEQ ID NO: 1 to SEQ ID NO: 27. In some embodiments, the inhibitor of EXT1 expression is an oligonucleotide having at least 75% identity with any one of sequences SEQ ID NO: 33 to SEQ ID NO: 53.

Within the scope of the instant invention, the expression “at least 75% identity” encompasses 75%, 76%, 77%, 78%, 79%, 80%, %, 81%, 82%, 83%, 84%, 85%, 86%, 87%, 88%, 89%, 90%, 91%, 92%, 93%, 94%, 95%, 96%, 97%, 98%, 99% and 100% identity.

The level of identity of 2 nucleic acid sequences may be performed by using any one of the known algorithms available from the state of the art.

Illustratively, the nucleic acid identity percentage may be determined using the CLUSTAL W software (version 1.83) the parameters being set as follows:
for slow/accurate alignments: (1) Gap Open Penalty: 15; (2) Gap Extension Penalty: 6.66; (3) Weight matrix: IUB;
for fast/approximate alignments: (4) K-tuple (word) size: 2; (5) Gap Penalty: 5; (6) No. of top diagonals: 5; (7) Window size: 4; (8) Scoring Method: PERCENT.

In some embodiments, the inhibitor of EXT1 expression is an oligonucleotide having at least 80% identity with any one of sequences SEQ ID NO: 1 to SEQ ID NO: 27 and SEQ ID NO: 33 to SEQ ID NO: 53. In certain embodiments, the inhibitor of EXT1 expression is an oligonucleotide having at least 85% identity with any one of sequences SEQ ID NO: 1 to SEQ ID NO: 27 and SEQ ID NO: 33 to SEQ ID NO: 53. In some embodiments, the inhibitor of EXT1 expression is an oligonucleotide having at least 90% identity with any one of sequences SEQ ID NO: 1 to SEQ ID NO: 27 and SEQ ID NO: 33 to SEQ ID NO: 53. In certain embodiments, the inhibitor of EXT1 expression is an oligonucleotide having at least 95% identity with any one of sequences SEQ ID NO: 1 to SEQ ID NO: 27 and SEQ ID NO: 33 to SEQ ID NO: 53. In some embodiments, the inhibitor of EXT1 expression is an oligonucleotide represented by any one of sequences SEQ ID NO: 1 to SEQ ID NO: 27 and SEQ ID NO: 33 to SEQ ID NO: 53.

In certain embodiments, the inhibitor of EXT1 expression is a shRNA having at least 75% identity with any one of sequences SEQ ID NO: 1 to SEQ ID NO: 24 and SEQ ID NO: 33 to SEQ ID NO: 53. In some embodiments, the inhibitor of EXT1 expression is a shRNA having at least 80% identity with any one of sequences SEQ ID NO: 1 to SEQ ID NO: 24 and SEQ ID NO: 33 to SEQ ID NO: 53. In certain embodiments, the inhibitor of EXT1 expression is a shRNA having at least 85% identity with any one of sequences SEQ ID NO: 1 to SEQ ID NO: 24 and SEQ ID NO: 33 to SEQ ID NO: 53. In some embodiments, the inhibitor of EXT1 expression is a shRNA having at least 90% identity with any one of sequences SEQ ID NO: 1 to SEQ ID NO: 24 and SEQ ID NO: 33 to SEQ ID NO: 53. In certain embodiments, the inhibitor of EXT1 expression is a shRNA having at least 95% identity with any one of sequences SEQ ID NO: 1 to SEQ ID NO: 24 and SEQ ID NO: 33 to SEQ ID NO: 53. In some embodiments, the inhibitor of EXT1 expression is a shRNA represented by any one of sequences SEQ ID NO: 1 to SEQ ID NO: 24 and SEQ ID NO: 33 to SEQ ID NO: 53.

In certain embodiments, the inhibitor of EXT1 expression is a shRNA represented by any one of sequences SEQ ID NO: 1, SEQ ID NO: 35, SEQ ID NO: 36, SEQ ID NO: 39, SEQ ID NO: 43, SEQ ID NO: 44, SEQ ID NO: 48, SEQ ID NO: 50 and SEQ ID NO: 52. In some embodiments, the inhibitor of EXT1 expression is a shRNA represented by sequence SEQ ID NO: 1. In certain embodiments, the inhibitor of EXT1 expression is a shRNA represented by any one of sequences SEQ ID NO: 35, SEQ ID NO: 36, SEQ ID NO: 39, SEQ ID NO: 43, SEQ ID NO: 44, SEQ ID NO: 48, SEQ ID NO: 50 and SEQ ID NO: 52. In some embodiments, the inhibitor of EXT1 expression is a shRNA represented by any one of sequences SEQ ID NO: 35, SEQ ID NO: 39, SEQ ID NO: 43, SEQ ID NO: 44, SEQ ID NO: 48 and SEQ ID NO: 52. In some embodiments, the inhibitor of EXT1 expression is a shRNA represented by SEQ ID NO: 35 or SEQ ID NO: 39.

In certain embodiments, the inhibitor of EXT1 expression is a siRNA having at least 75% identity with any one of sequence SEQ ID NO: 25 to SEQ ID NO: 27. In some embodiments, the inhibitor of EXT1 expression is a siRNA having at least 80% identity with any one of sequence SEQ ID NO: 25 to SEQ ID NO: 27. In certain embodiments, the inhibitor of EXT1 expression is a siRNA having at least 85% identity with any one of sequence SEQ ID NO: 25 to SEQ ID NO: 27. In some embodiments, the inhibitor of EXT1 expression is a siRNA having at least 90% identity with any one of sequence SEQ ID NO: 25 to SEQ ID NO: 27. In certain embodiments, the inhibitor of EXT1 expression is a siRNA having at least 95% identity with any one of sequence SEQ ID NO: 25 to SEQ ID NO: 27. In some embodiments, the inhibitor of EXT1 expression is a siRNA represented by any one of sequence SEQ ID NO: 25 to SEQ ID NO: 27.

In some aspect, the invention further relates to a use of an inhibitor of EXT1 activity for the production of a biological entity in a cell.

In some embodiments, the inhibitor of EXT1 activity comprises an oligopeptide, a polypeptide or a chemical compound.

Within the scope of the instant invention, the term “oligopeptide” refers to a linear polymer of less than 50 amino acids linked together by peptide bonds. Within the scope of the instant invention, the term “polypeptide” refers to a linear polymer of at least 50 amino acids linked together by peptide bonds.

In some embodiments, suitable oligopeptides and chemical compounds according to the invention interfere with the enzymatic property of EXT1, in particular, with the catalytic site of EXT1.

In practice, said inhibitor of the EXT1 activity is a polypeptide, preferably an EXT1 binding compound selected in a group comprising an antibody, an antibody fragment, an afucosylated antibody, a diabody, a triabody, a tetrabody, a nanobody, and an analog thereof.

As used herein, an “antibody”, also referred to as immunoglobulins (abbreviated “Ig”), is intended to refer to a gamma globulin protein that is found in blood or other bodily fluids of vertebrates, and is used by the immune system to identify and neutralize foreign objects, such as bacteria and viruses. Antibodies consist of two pairs of polypeptide chains, called heavy chains and light chains that are arranged in a Y-shape. The two tips of the Y are the regions that bind to antigens and deactivate them. The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

As used herein, an “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments. One may refer to a “functional fragment or analog” of an antibody, which is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc[epsilon]RI. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

As used herein, an “afucosylated antibody” refers to an antibody lacking core fucosylation. As a matter of fact, nearly all IgG-type antibodies are N-glycosylated in their Fc moiety. Typically, a fucose residue is attached to the first N-acetylglucosamine of these complex-type N-glycans. In other words, an “afucosylated antibody” refers to an antibody that does not possess N-glycans.

As used herein, the term “diabody” refers to a small antibody fragment prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described in more details in, e.g., EP 404,097; WO 93/11161; and Hollinger et al., Proc. Natl. Acad. Sci. USA, 90:6444-6448 (1993).

As used herein, a “triabody” is intended to refer to an antibody that has three Fv heads, each consisting of a VH domain from one polypeptide paired with the VL domain from a neighboring polypeptide.

As used herein, a “nanobody” refers to a functional antibody that consists of heavy chains only and therefore lack light chains. These heavy-chain only antibodies contain a single variable domain (VHH) and two constant domains (CH2, CH3).

In some embodiment, the biological entity is a recombinant biological entity. In certain embodiments, the biological entity is selected in a group comprising a recombinant polypeptide and/or a viral particle.

In some embodiments, the recombinant polypeptide may include, without being limited to, a recombinant polypeptide of therapeutic interest, such as, e.g., an antibody, hormone, interferon, interleukin, growth factor, tumor necrosis factor, blood clotting factor, thrombolytic factor, and enzyme. Illustratively, recombinant polypeptide of interest may be chosen in the non-limitative list comprising epoietin alpha, factor VIIa, factor VIII, factor IX, insulin, interferon alpha 2b, interferon beta 1a, interferon beta 1b, somatropin. In some embodiments, the recombinant polypeptide may be of viral origin, such as, e.g., GAG and POL polypeptides from the HIV-1 virus; gp-120 and gp-41 glycoproteins of HIV virus; Rev protein of HIV-1; vesicular stomatitis virus glycoprotein (VSV-G).

In some embodiments, the viral particle is preferably selected in a group comprising an adenovirus, an adeno-associated virus (AAV), an alphavirus, a baculovirus, a herpes simplex virus, a lentivirus, a non-integrative lentivirus, a retrovirus, vaccinia virus.

In some embodiments, the cell is a eukaryote cell.

Within the scope of the invention, a “eukaryote cell” encompasses a yeast, an algae cell, a plant cell, an animal cell, such as, e.g., an insect cell, a mammal cell, including a human cell.

In certain embodiments, the eukaryotic cell is an insect cell, such as, e.g., S2, Sf21, Sf9 or High Five cell.

In some preferred embodiments, the eukaryotic cell is a mammal cell, preferably a human or hamster cell.

In certain embodiments, a target cell and/or a host cell according to the instant invention may encompass, without limitation, a cell of the central nervous system, an epithelial cell, a muscular cell, an embryonic cell, a germ cell, a stem cell, a progenitor cell, a hematopoietic stem cell, a hematopoietic progenitor cell, an induced Pluripotent Stem Cell (iPSC).

In some particular embodiments, the target cell and/or the host cell is not a stem cell, a progenitor cell, a germinal cell or an embryonic cell.

In some embodiments, the target cell and/or the host cell may belong to a tissue selected in a group comprising a muscle tissue, a nervous tissue, a connective tissue, and an epithelial tissue.

In some embodiments, the target cell and/or the host cell may belong to an organ selected in a group comprising a bladder, a bone, a brain, a breast, a central nervous system, a cervix, a colon, an endometrium, a kidney, a larynx, a liver, a lung, an esophagus, an ovarian, a pancreas, a pleura, a prostate, a rectum, a retina, a salivary gland, a skin, a small intestine, a soft tissue, a stomach, a testis, a thyroid, an uterus, a vagina.

In some embodiments, the eukaryote cell is a mammal cell, such as, e.g., CHO, COS-7 HEK293, HeLa, lymphocyte cell.

In practice, these cells may originate from commercially available cell lines.

In some embodiments, the inhibitor of EXT1 expression and/or activity is introduced into the host cell by transfection.

In practice, transfection may be performed according to the methods known in the state of the art, or methods adapted therefrom. Illustratively, these methods include chemical transfection, gene gun, electroporation, sonoporation, magnetofection, and viral-mediated transfection.

In some embodiments, transfection is performed chemically, in particular by the mean of calcium phosphate, cationic lipids, dendrimers, liposomes, polycation, polymers and/or nanoparticles. In some embodiments, chemical transfection includes the use of calcium phosphate, polyethylenimine or lipofectamine.

In certain embodiments, transfection is performed by the mean of a viral vector, in particular a retrovirus, a lentivirus, an adenovirus, an adeno-associated virus and combination thereof.

In practice, assessment of inhibition of EXT1 expression and/or activity in a host cell may be performed by any suitable method known in the state of the art, or a method adapted therefrom.

In some embodiments, inhibition of EXT1 expression may be assessed at the nucleic acid (mRNA) level. A non-limitative example of these methods may encompass a real-time RT-PCR (qPCR) analysis of RNA extracted from cultured cells with specific primers, RNA sequencing (RNASeq).

In certain embodiments, inhibition of EXT1 activity may be assessed at the protein level. A non-limitative example of these methods may encompass an immunofluorescence analysis with markers-specific antibodies, Western blotting, ELISA, Fluorescent activated cell sorting (FACS), or any functional protein activity assay. For an example of functional EXT1 activity assay, one may refer to the assay disclosed by McCormick et al. (PNAS USA, 2000, Jan 18; 97(2):668-673). In some embodiments, the EXT1 glycosyltransferase enzymatic activity may be assessed by the mean of the commercial glycosyltransferase Activity Kit (R&D Systems®).

In some aspect, the invention also pertains to a use of a cell having at least depleted EXT1 expression and/or activity for the production of a biological entity.

In certain embodiments, the cell is a eukaryote cell.

In some embodiments, the biological entity is selected in a group comprising a recombinant polypeptide and/or a viral particle.

In certain embodiments, the cell comprises a partial or total knockout of the EXT1 gene.

In practice, methods for achieving partial or total knockout of a gene of interest are known from a skilled in the art. Illustratively, partial or total knockout of a gene of interest may be performed by gene editing, e.g., by the CRISPR or TALEN method.

In some embodiments, the at least depleted EXT1 expression and/or activity is obtained by the treatment of said cell with an inhibitor of EXT1 expression and/or activity.

In some embodiments, said inhibitor of the EXT1 expression and/or activity is selected from a group comprising an oligonucleotide, an aptamer, an oligopeptide, a polypeptide, a chemical compound and an analog thereof.

In certain embodiments, said inhibitor of the EXT1 expression is selected in a group comprising an oligonucleotide having at least 75% identity with any one of sequences SEQ ID NO: 1 to SEQ ID NO: 27 and SEQ ID NO: 33 to SEQ ID NO: 53, preferably an oligonucleotide represented by any one of sequences SEQ ID NO: 1 to SEQ ID NO: 27 and SEQ ID NO: 33 to SEQ ID NO: 53.

In practice, the production of biological entity by a cell depleted in EXT1 comprises the culture of said cell in a culture medium. As used herein, the term “culture medium” refers to the generally accepted definition in the field of cellular biology, i.e., any medium suitable for promoting the growth of the cells of interest.

In some embodiments, a suitable culture medium may include a chemically defined medium, i.e., a nutritive medium only containing specified components, preferably components of known chemical structure.

In some embodiments, a chemically defined medium may be a serum-free and/or feeder-free medium. As used herein, a “serum-free” medium refers to a culture medium containing no added serum. As used herein, a “feeder-free” medium refers to a culture medium containing no added feeder cells.

A culture medium for use according to the invention may be an aqueous medium that may include a combination of substances such as one or more carbon/energy sources, amino acids, vitamins, inorganic salts, trace elements, reducing agents, buffering agents, lipids, nucleosides, antibiotics, antimycotics, hormones, cytokines, and growth factors.

In practice, suitable carbon/energy sources include D-glucose, pyruvate, lactate, ATP, creatine, creatine phosphate, and a mix thereof. As used herein, “amino acids” encompass L-alanine; L-arginine; L-asparagine; L-aspartic acid; L-cysteine; L-cystine; L-glutamine; L-glutamic acid; glycine; L-histidine; L-isoleucine; L-leucine; L-lysine; L-methionine; L-phenylalanine; L-proline; L-serine; taurine; L-threonine; L-tryptophan; L-tyrosine; L-valine. As used herein, “vitamins” encompass biotin (vitamin H); D-calcium-pantothenate; choline chloride; folic acid (vitamin B9); myo-inositol; nicotinamide; pyridoxal (vitamin B6); riboflavin (vitamin B2); thiamine (vitamin B1); cobalamin (vitamin B12); acid ascorbic; α-tocopherol (vitamin E) and a combination of two or more vitamins thereof. Non-limitative examples of suitable inorganic salts include calcium bromide, calcium chloride, calcium phosphate, calcium nitrate, calcium nitrite, calcium sulphate, magnesium bromide, magnesium chloride, magnesium sulphate, potassium bicarbonate, potassium bromide, potassium chloride, potassium dihydrogen phosphate, potassium disulphate, di- potassium hydrogen phosphate, potassium nitrate, potassium nitrite, potassium sulphite, potassium sulphate, sodium bicarbonate, sodium bromide, sodium chloride, sodium disulphate, sodium hydrogen carbonate, sodium dihydrogen phosphate, di-sodium hydrogen phosphate, sodium sulphate and a mix thereof. In practice, trace elements may include copper (Cu), iron (Fe), manganese (Mn), selenium (Se) and zinc (Zn). Non-limitative examples of antibiotics include ampicillin, kanamycin, penicillin, streptomycin and tetracycline. One example of antimycotics includes amphotericin B. As used herein, “hormones” include insulin; 17β-estradiol; human transferrin; progesterone; corticosterone; triiodothyronine (T3) and a mix thereof.

Examples of suitable culture media include, without being limited to RPMI medium, William’s E medium, Basal Medium Eagle (BME), Eagle’s Minimum Essential Medium (EMEM), Minimum Essential Medium (MEM), Dulbecco’s Modified Eagles Medium (DMEM), Ham’s F-10, Ham’s F-12 medium, Kaighn’s modified Ham’s F-12 medium, DMEM/F-12 medium, and McCoy’s 5A medium, which may be further supplemented with any one of the above-mentioned substances.

In practice, the culture parameters such as the temperature, the pH, the salinity, and the levels of O₂ and CO₂ are adjusted accordingly to the standards established in the state of the art.

Illustratively, the temperature for culturing the cells according to the invention may range from about 20° C. to about 42° C., preferably from about 25° C. to about 40° C.

Within the scope of the invention, the expression “from about 20° C. to about 42° C.” encompasses 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C. and 42° C.

In some embodiments, the level of CO₂ during the course of culture is maintained constant and ranges from about 1% to about 10%, preferably from about 2.5% to about 7.5%.

Within the scope of the instant invention, the expression “from about 1% to about 10%” encompasses 1%, 2%, 3%, 4%, 5%, 6%, 7%, 8%, 9% and 10%.

In certain embodiments, the culture medium is changed at least every other day, preferably every day, during the course of the culture.

In practice, the culture medium is removed, the cells may be washed once or twice with fresh culture medium and a fresh culture medium is provided to the cells.

In another aspect, the invention relates to a method for the production of a biological entity in a cell, said method comprising the steps of:
a) providing a cell population having at least depleted EXT1 expression and/or activity;
b) transfecting the cell population of step a) with an oligonucleotide encoding the biological entity, preferably a polypeptide or a viral particle.

In some embodiments, the method further comprises the step of:

c) culturing the transfected cell population obtained at step b) in a suitable culture medium, so as to synthesize the polypeptide or the viral particle.

In certain embodiments, the method further comprises the step of:

d) extracting and/or purifying the synthesized polypeptide or viral particle.

Another aspect of the invention further pertains to a method for the production of a biological entity in a cell, said method comprising the steps of:
a) providing a cell population;
b) transfecting the cell population of step a) with an oligonucleotide encoding the biological entity, preferably a polypeptide or a viral particle,
c) inhibiting EXT1 expression and/or activity in the said cell by using an EXT1 inhibitor as defined in the instant invention.

In some embodiments, the method further comprises the step of:

d) culturing the transfected cell population obtained at step b) in a suitable culture medium, so as to synthesize the polypeptide or the viral particle.

In certain embodiments, the method further comprises the step of:

e) extracting and/or purifying the synthesized polypeptide or viral particle.

In practice, extraction and/or purification of the synthesized biological entity of interest may be performed according to any suitable method known from the state in the art, or a method adapted therefrom. Illustratively, mechanical/physical and/or chemical methods may be implemented. Non-limitative examples of mechanical/physical methods include glass beads, pressure (press), ultrasounds (sonication). Non-limitative examples of chemical methods include detergent-mediated (e.g., CHAPS, NP-40, SDS, Triton X-100, Tween-20 or Tween-80), or detergent and protease-mediated, cell lysis.

Polypeptide of interest may be extracted by the mean of commercial kits, such as, e.g., ProteoExtract® kits (Millipore®), ProteoPrep® kits (Millipore®), ReadyPrep® Protein Extraction kit (BioRad®).

In certain embodiments, oligonucleotide encoding the biological entity is selected from the group comprising, or consisting of, a plasmid, a cosmid or a bacterial artificial chromosome.

As used herein, “plasmid” refers to a small extra-genomic DNA molecule, most commonly found as circular double stranded DNA molecules that may be used as a cloning vector in molecular biology, to make and/or modify copies of DNA fragments up to about 15 kb (i.e., 15,000 base pairs). Plasmids may also be used as expression vectors to produce large amounts of proteins of interest encoded by a nucleic acid sequence found in the plasmid downstream of a promoter sequence.

As used herein, the term “cosmid” refers to a hybrid plasmid that contains cos sequences from Lambda phage, allowing packaging of the cosmid into a phage head and subsequent infection of bacterial cell wherein the cosmid is cyclized and can replicate as a plasmid. Cosmids are typically used as cloning vector for DNA fragments ranging in size from about 32 to 52 kb.

As used herein, “bacterial artificial chromosome” or “BAC” refers to an extra-genomic nucleic acid molecule based on a functional fertility plasmid that allows the even partition of said extra-genomic DNA molecules after division of the bacterial cell. BACs are typically used as cloning vector for DNA fragment ranging in size from about 150 to 350 kb.

In practice, the oligonucleotide encoding the biological entity may be in the form of a plasmid, in particular resulting from the cloning of a nucleic acid of interest into a nucleic acid vector. In some embodiments, non-limitative suitable nucleic acid vectors are pBluescript vectors, pET vectors, pETduet vectors, pGBM vectors, pBAD vectors, pUC vectors. In one embodiment, the plasmid is a low copy plasmid. In one embodiment, the plasmid is a high copy plasmid.

In some embodiments, the oligonucleotide encoding the biological entity may also encodes the EXT1 expression inhibitor, in particular an EXT1 expression inhibitor selected in the group of miRNA, guide RNA, siRNA, shRNA.

In certain embodiments, the oligonucleotide encoding the biological entity encodes a recombinant protein and a shRNA or a siRNA that inhibits the expression of EXT1.

In certain embodiments, the polypeptide of interest may comprise a tag-domain, for the ease of purification. Non-limiting examples of tag-domains suitable for the invention may be selected in a group comprising a FLAG-tag, GST-tag, Halo-Tag, His-tag, MBP-tag, Snap-Tag, SUMO-tag and a combination thereof.

Recombinant proteins produced in an EXT1-depleted cell according to the invention may be for use for human or veterinary therapy, such as, e.g., preventing and/or treating an auto-immune disease, a cancer, an infectious disease, an inflammatory disease, a metabolic disease, a neurogenerative disease.

Non-limitative examples of auto-immune diseases include Addison’s disease, auto-immune vasculitis, celiac disease, Graves’ disease, Hashimoto’s thyroiditis, inflammatory bowel disease (IBD; including Crohn’s disease and Ulcerative disease), multiple sclerosis (MS), myasthenia gravis, pernicious anemia, psoriasis (or psoriatic arthritis), rheumatoid arthritis (RA), Sjögren’s syndrome, systemic lupus erythematosus (SLE) and type 1 diabetes.

Non-limitative examples of cancer encompass bladder cancer, bone cancer, brain cancer, breast cancer, cancer of the central nervous system, cancer of the cervix, cancer of the upper aero digestive tract, colorectal cancer, endometrial cancer, germ cell cancer, glioblastoma, Hodgkin lymphoma, kidney cancer, laryngeal cancer, leukemia, liver cancer, lung cancer, myeloma, nephroblastoma (Wilms tumor), neuroblastoma, non-Hodgkin lymphoma, esophageal cancer, osteosarcoma, ovarian cancer, pancreatic cancer, pleural cancer, prostate cancer, retinoblastoma, skin cancer (including melanoma), small intestine cancer, soft tissue sarcoma, stomach cancer, testicular cancer and thyroid cancer. Non-limitative examples of the infectious disease include Anaplasmosis; Anthrax; Babesiosis; Botulism; Brucellosis; Burkholderia mallei infection (glanders); Burkholderia pseudomallei infection (melioidosis); Campylobacteriosis; Carbapenem-resistant Enterobacteriaceae infection (CRE); Chancroid; Chikungunya infection; Chlamydia infection; Ciguatera; Clostridium difficile infection; Clostridium perfringens infection (Epsilon Toxin); Coccidioidomycosis fungal infection (Valley fever); Creutzfeldt-Jacob Disease, transmissible spongiform (CJD); Cryptosporidiosis; Cyclosporiasis; Dengue Fever; Diphtheria; E. Coli infection; Eastern Equine Encephalitis (EEE); Ebola Hemorrhagic Fever (Ebola); Ehrlichiosis; Arboviral or parainfectious encephalitis; Non-polio enterovirus infection; D68 enterovirus infection, (EV-D68); Giardiasis; Gonococcal infection (Gonorrhea); Granuloma inguinale; Type B Haemophilus influenza disease, (Hib or H-flu); Hantavirus pulmonary syndrome (HPS); Hemolytic uremic syndrome (HUS); Hepatitis A (Hep A); Hepatitis B (Hep B); Hepatitis C (Hep C); Hepatitis D (Hep D); Hepatitis E (Hep E); Herpes; Herpes zoster, zoster VZV (Shingles); Histoplasmosis; Human Immunodeficiency Virus/AIDS (HIV/AIDS); Human Papillomavirus (HPV); Influenza (Flu); Lead poisoning; Legionellosis (Legionnaires Disease); Leprosy (Hansens Disease); Leptospirosis; Listeriosis; Lyme Disease; Lymphogranuloma venereum infection (LVG); Malaria; Measles; Viral meningitis; Meningococcal disease; Middle East respiratory syndrome coronavirus (MERS-CoV); Mumps; Norovirus; Paralytic shellfish poisoning; Pediculosis (lice, head and body lice); Pelvic inflammatory disease (PID); Pertussis; Bubonic, septicemic or pneumonic plague,; Pneumococcal disease; Poliomyelitis (Polio); Psittacosis; Pthiriasis (crabs; pubic lice infestation); Pustular rash diseases (small pox, monkeypox, cowpox); Q-Fever; Rabies; Ricin poisoning; Rickettsiosis (Rocky Mountain Spotted Fever); Rubella, including congenital rubella (German Measles); Salmonellosis gastroenteritis infection; Scabies infestation; Scombroid; Severe acute respiratory syndrome (SARS); Shigellosis gastroenteritis infection; Smallpox; Methicillin-resistant Staphylococcal infection (MRSA); Staphylococcal food poisoning; Vancomycin intermediate Staphylococcal infection (VISA); Vancomycin resistant Staphylococcal infection (VRSA); Streptococcal disease, Group A; Streptococcal disease, Group B; Streptococcal toxic-shock syndrome (STSS); Primary, secondary, early latent, late latent or congenital syphilis; Tetanus infection (Lock Jaw); Trichinosis; Tuberculosis (TB); Latent tuberculosis (LTBI); Tularemia (rabbit fever); Typhoid fever, Group D; Typhus; Vaginosis; Varicella (chickenpox); Vibrio cholerae infection (Cholera); Vibriosis (Vibrio); Viral hemorrhagic fever (Ebola, Lassa, Marburg); West Nile virus infection; Yellow Fever; Yersinia infection and Zika virus infection.

Non-limitative examples of inflammatory diseases include active hepatitis, asthma, chronic peptic ulcer, Crohn’s disease, dermatitis, periodontitis, rheumatoid arthritis, sinusitis, tuberculosis and ulcerative colitis.

Non-limitative examples of metabolic diseases include abnormal lipid metabolism, alcoholic fatty liver disease, atherosclerosis, dyslipidemia, glucose intolerance, hepatic steatosis, hyperglycemia, hypertension, insulin-deficiency, insulin-resistance related disorders, irritable bowel syndrome (IBS), metabolic syndrome, non-alcoholic fatty liver disease, obesity and type 2 diabetes.

Non-limitative examples of neurodegenerative disease encompass Alzheimer’s disease, Amyotrophic lateral sclerosis, Down’s syndrome, Friedreich’s ataxia, Huntington’s disease, Lewy body disease, Parkinson’s disease and Spinal muscular atrophy.

In some embodiment, the recombinant protein is selected in a group comprising human therapeutic antibodies, murine therapeutic antibodies, chimeric therapeutic antibodies and humanized therapeutic antibodies.

Non-limitative examples of human therapeutic antibodies that may be produced in an EXT1-depleted cell according to the invention encompass Panitumumab. Actoxumab, Adalimumab, Adecatumumab, Alirocumab, Anifrolumab, Atinumab, Atorolimumab, Belimumab, Bertilimumab, Bezlotoxumab, Bimagrumab, Briakinumab, Brodalumab, Canakinumab, Carlumab, Cixutumumab, Conatumumab, Daratumumab, Denosumab, Drozitumab, Duligotumab, Dupilumab, Dusigitumab, Efungumab, Eldelumab, Enoticumab, Evolocumab, Exbivirumab, Fasinumab, Fezakinumab, Figitumumab, Flanvotumab, Foralumab, Foravirumab, Fresolimumab, Fulranumab, Ganitumab, Gantenerumab, Glembatumumab vedotin, Golimumab, Guselkumab, Icrucumab, Inclacumab, Intetumumab, Ipilimumab, Iratumumab, Lerdelimumab, Lexatumumab, Libivirumab, Lirilumab, Lucatumumab, Mapatumumab, Mavrilimumab, Metelimumab, Morolimumab, Namilumab, Narnatumab, Nebacumab, Necitumumab, Nesvacumab, Nivolumab, Ofatumumab, Olaratumab, Orticumab, Oxelumab, Panitumumab, Panobacumab, Parsatuzumab, Patritumab, Placulumab, Pritumumab, Radretumab, Rafivirumab, Ramucirumab, Raxibacumab, Regavirumab, Rilotumumab, Robatumumab, Roledumab, Sarilumab, Secukinumab, Seribantumab, Sevirumab, Sirukumab, Stamulumab, Tabalumab, Teprotumumab, Ticilimumab (= tremelimumab), Tovetumab, Tralokinumab, Tremelimumab, Tuvirumab, Urelumab, Ustekinumab, Vantictumab, Vesencumab, Votumumab, Zalutumumab, Zanolimumab, Ziralimumab.

Non-limitative examples of murine therapeutic antibodies that may be produced in an EXT1-depleted cell according to the invention include Abagovomab, Afelimomab, Anatumomab mafenatox, Blinatumomab, Detumomab, Dorlimomab aritox, Edobacomab, Edrecolomab, Elsilimomab, Enlimomab pegol, Epitumomab cituxetan, Faralimomab, Gavilimomab, Ibritumomab tiuxetan, Imciromab, Inolimomab, Lemalesomab, Maslimomab, Minretumomab, Mitumomab, Moxetumomab pasudotox, Muromonab-CD3, Nacolomab tafenatox, Naptumomab estafenatox, Nerelimomab, Odulimomab, Oregovomab, Pemtumomab, Racotumomab, Solitomab, Taplitumomab paptox, Telimomab aritox, Tenatumomab, Tositumomab, Vepalimomab and Zolimomab aritox.

Non-limitative examples of chimeric therapeutic antibodies that may be produced in an EXT1-depleted cell according to the invention encompass Abciximab, Amatuximab, Basiliximab, Bavituximab, Brentuximab vedotin, Cetuximab, Clenoliximab, Ecromeximab, Ensituximab, Futuximab, Galiximab, Girentuximab, Gomiliximab, Indatuximab ravtansine, Infliximab, Keliximab, Lumiliximab, Pagibaximab, Priliximab, Pritoxaximab, Rituximab, Setoxaximab, Siltuximab, Teneliximab, Ublituximab, Vapaliximab, Volociximab and Zatuximab.

Non-limitative examples of humanized therapeutic antibodies that may be produced in an EXT1-depleted cell according to the invention include Afutuzumab, Alacizumab pegol, Alemtuzumab, Anrukinzumab, Apolizumab, Aselizumab, Atlizumab (=tocilizumab), Bapineuzumab, Benralizumab, Bevacizumab, Bivatuzumab mertansine, Blosozumab, Cantuzumab mertansine, Cantuzumab ravtansine, Caplacizumab, Cedelizumab, Certolizumab pegol, Citatuzumab bogatox, Clazakizumab, Clivatuzumab tetraxetan, Concizumab, Crenezumab, Dacetuzumab, Daclizumab, Dalotuzumab, Demcizumab, Eculizumab, Efalizumab, Elotuzumab, Enavatuzumab, Enokizumab, Epratuzumab, Erlizumab, Etaracizumab, Etrolizumab, Farletuzumab, Felvizumab, Ficlatuzumab, Fontolizumab, Gemtuzumab ozogamicin, Gevokizumab, Ibalizumab, Imgatuzumab, Inotuzumab ozogamicin, Itolizumab, Ixekizumab, Labetuzumab, Lambrolizumab, Lampalizumab, Lebrikizumab, Ligelizumab, Lintuzumab, Lodelcizumab, Lorvotuzumab mertansine, Margetuximab, Matuzumab, Mepolizumab, Milatuzumab, Mogamulizumab, Motavizumab, Natalizumab, Nimotuzumab, Ocaratuzumab, Ocrelizumab, Olokizumab, Omalizumab, Onartuzumab, Oportuzumab monatox, Ozanezumab, Ozoralizumab, Palivizumab, Pascolizumab, Pateclizumab, Perakizumab, Pertuzumab, Pexelizumab, Pidilizumab, Pinatuzumab vedotin, Polatuzumab vedotin, Ponezumab, Quilizumab, Ranibizumab, Reslizumab, Romosozumab, Rontalizumab, Rovelizumab, Ruplizumab, Samalizumab, Sibrotuzumab, Sifalimumab, Simtuzumab, Siplizumab, Solanezumab, Sonepcizumab, Sontuzumab, Suvizumab, Tacatuzumab tetraxetan, Tadocizumab, Talizumab, Tanezumab, Tefibazumab, Teplizumab, Tildrakizumab, Tigatuzumab, Tocilizumab (=atlizumab), Toralizumab, Trastuzumab, Tregalizumab, Tucotuzumab celmoleukin, Urtoxazumab, Vatelizumab, Vedolizumab, Veltuzumab, Visilizumab and Vorsetuzumab mafodotin.

### BRIEF DESCRIPTION OF THE DRAWINGS

FIGS. 1A-1B are a combination of histograms showing the average Pearson’s correlation coefficient of indicated markers and EXT1 protein (A) in Cos7 cells transiently expressing SYFP2-EXT1 and endogenous markers calnexin, PDIA3, GM130; or (B) in Cos7 cells co-expression of SYFP2-EXT1 and the markers Lnp1, ATL1, RTN4a (n = 12).

FIGS. 2A-2D are photographs showing: (A) the efficient depletion of EXT1 protein by shRNA. HSP70 protein is used as a loading control; Cos7 cells stably expressing indicated markers: (B) Lnp1, (C) ATL1, (D) RTN4a. Boxed regions magnified show ER tubular network. Scale bar, 4 µm.

FIGS. 3A-3B are a combination of photographs and graph showing the localization of mEmerald-Sec61b in Cos7 cells expressing shCTRL (upper panels) or shEXT1 (lower panels): (A) original image; (B) skeleton.

FIG. 4 is a graph showing the ER metrics analysis of cells co-expressing mEmerald-Sec61b and shCTRL or shEXT1. The boxplot indicates the mean, and whiskers show the minimum, and maximum values (n =24). Tubule mean length is expressed in µm.

FIG. 5 is a combination of photographs showing live imaging of activated PA-GFP-KDEL in Cos7 cells expressing shCTRL (upper panel) or shEXT1 (lower panel). Scale bar, 5 µm.

FIG. 6 is a graph showing the mean normalized fluorescence intensity (a.u.) after the addition of biotin in HeLa cells expressing shCTRL (circles) or shEXT1 (squares).

FIG. 7 is a graph showing the average Pearson’s correlation coefficient of VSVG-GFP localized at membranes of HeLa cells, at the indicated time points (n = 10). Mean number + SD. One-way ANOVA: **p<0.01; ***p<0.001; n.s., not significant.

FIG. 8 is a combination of photographs showing TEM analysis of trans-Golgi area of HeLa cells expressing shCTRL (left panel) or shEXT1 (right panel). Higher magnification of the boxed area is shown. Scale bar, 1 µm.

FIG. 9 is a graph showing the number of secretion vesicles in the trans-Golgi area quantified based on TEM images from FIG. 8 (n = 17-18). HeLa cells expressing shCTRL (dark grey) or shEXT1 (light grey). Mean number + SD. One-way ANOVA: ****p<0.0001.

FIGS. 10A-10B is a combination of photographs showing TEM analysis of Golgi apparatus in HeLa cells expressing shCTRL (A) or shEXT1 (B). Higher magnification of the boxed area is shown. Scale bar, 500 nm.

FIGS. 11A-11B is a combination of photographs showing schematic representations of the Golgi apparatus in cells expressing shCTRL (A) or shEXT1 (B), as used for the statistical analysis of the different parameters (length, number of cisternae/stack).

FIG. 12 is a graph showing the number of Golgi cisternae/stack in HeLa cells expressing shCTRL (dark grey) or shEXT1 (light grey). Mean number + SD. (n = 18-21). Oneway ANOVA: ****p<0.0001.

FIG. 13 is a graph showing the maximum length of individual Golgi cisternae (nm) in HeLa cells expressing shCTRL (dark grey) or shEXT1 (light grey). Mean length + SD. (n = 18-21). Oneway ANOVA: ****p<0.0001.

FIGS. 14A-14B is a combination of photographs showing TEM analysis of the ultrastructure ER of HeLa cells expressing shCTRL (A) or shEXT1 (B). Scale bar, 2 µm.

FIGS. 15A-15C is a combination of photographs and graph showing the TEM analysis of ER morphology in HEK293 cells expressing shCTRL (A) or shEXT1 (B) (Scale bar, 2 µm); and (C), the relative mRNA expression level of EXT1 gene was analyzed by qPCR in HEK293 cells expressing shCTRL (dark grey) or shEXT1 (light grey). One-way ANOVA: ****p<0.0001.

FIGS. 16A-16B is a combination of graphs showing the quantitative proteomic analysis of microsomes. (A), pie chart illustrating the number of up- and down-regulated proteins; (B), heatmap shows the PSMs number of 23 ER integral proteins.

FIGS. 17A-17B is a combination of graphs showing the N-glycans profiles of microsomes isolated from HeLa cells expressing shCTRL (dark grey) or shEXT1 (light grey). (A), graph showing the relative abundance of fucosylated, mono-fucosylated and difucosylated glycans; (B), graph showing the relative abundance of sialylated, mono-sialylated, di-sialylated, and 3+sialytated N-glycans.

FIGS. 18A-18B is a combination of graphs showing the glycomics analysis of microsomes. (A) bars indicate the fold change of the total N- and O- glycans intensity; (B) graph representing the relative abundance of each N-glycan in microsomes; the variations are plotted by N-glycan mass (m/z).

FIGS. 19A-19B is a combination of photographs showing the TEM analysis of HeLa cells expressing shCTRL (A) or shEXT1 (B). Scale bar, 2 µm.

FIGS. 20A-20B is a combination of graphs showing the schematic representation of ER-mitochondria and ER-nuclear envelope contact sites in Hela cells expressing shCTRL (A) or shEXT1 (B).

FIGS. 21A-21B is a combination of graphs showing the quantification of contact sites (n = 10-18), as expressed as the number of contact sites in ER per nuclear envelop (A) or the percentage of mitochondria/ER contact sites (B) in HeLa cells expressing shCTRL or shEXT1.

FIG. 22 is a graph showing the quantification of the total rough Endoplasmic reticulum (RER) length (nm)/cell (n = 10), in HeLa cells expressing shCTRL or shEXT1. Boxplot indicates the mean and whiskers show the minimum and maximum values. One-way ANOVA: ****p<0.0001.

FIG. 23 is a graph showing the fractional contribution from ¹³C₆-Glucose to TCA metabolites (n = 3) in cells expressing shCTRL (dark grey) or shEXT1 (light grey). Mean number + SD is plotted. One-way ANOVA: ****p<0.0001.

FIG. 24 is a graph showing the comparison of mass isotopomer distribution (MID) of citrate derivatives in HEK293 cells expressing shCTRL (dark grey) or shEXT1 (light grey). Mean number + SD is plotted. One-way ANOVA: ***p<0.001; ****p<0.0001; n.s., not significant.

FIG. 25 is a graph showing the metabolomic analysis from ¹³C₅-Pentose of pentose phosphate pathway metabolites in HEK293 cells. Fold change in the abundance of the metabolites in shEXT1/shCTRL. One-way ANOVA: *p<0.05; ****p<0.0001; n.s., not significant.

FIG. 26 is a graph showing the cell abundance from ¹³C₆-glucose of pentose phosphate pathway metabolites. Fold change in the abundance of the metabolites in shEXT1/shCTRL. Mean number + SD is plotted. One-way ANOVA: *p<0.05; ****p<0.0001; n.s., not significant.

FIG. 27 is a graph showing the percentage of energy charge. Mean number + SD is plotted. One-way ANOVA: ***p<0.001.

FIG. 28 is a graph showing the relative production of lentiviral VsVg viral particles in cells expressing shCTRL or shEXT1.

FIG. 29 is a graph showing the relative production of AAV2 viral particles in HEK293 cells expressing shCTRL or shEXT1.

FIG. 30 is a graph showing the relative production of recombinant NOTCH protein in HEK293 cells expressing shCTRL or shEXT1.

FIG. 31 is a graph showing the relative production of luciferase from a VsVg lentivirus in HeLa cells expressing shCTRL or shEXT1.

FIGS. 32A-32C is a set of photographs showing the expression of EXT1 profile in different HEK293 cell lines transfected with shRNA constructs by Western blot. The numbers correspond to shRNA constructs in Table 4. (A): shRNAs#1, 2, 3, 6, 7 and C; (B): shRNAs#10, 13, 16, 17, 18, 19 and C; (C): shRNAs#4, 5, 8, 9, 11, 12, 15 and C. “C” refers to control shRNA. Stained proteins (human EXT1) and GAPDH are indicated. Arrows indicated EXT1 knock down compared to the control (C).

FIGS. 33A-33B is a set of graphs showing (A) the nano-luciferase activities after transduction of HEK293 cell lines knocked down for EXT1 using indicated shRNA sequences; and (B) the fluorescence intensities following transduction using AAV2-GFP virus. The numbers of shRNA correspond to Table 4. Statistical analysis of three independent experiments: One-way ANOVA: ****p<0.0001; ***p<0.001; **p<0.01; ****p<0.1; ns: not significant.

### EXAMPLES

The present invention is further illustrated by the following examples.

### Example 1: Depletion of EXT1 Results in an Altered Endoplasmic Reticulum (ER)

### 1- Material and Methods

### 1.1- Plasmids

HA-SEC13 pRK5 (#46332), mEmerald-Sec61b-C1 (#90992), pEGFP-SEC16b (#66607), pEGFP-SEC23A(66609), Str-KDEL-TNF-SBP-mCherry (#65279), b4GALT1-pmTirquoise2-N1 (#36205) constructs were obtained from Addgene®. ts045-VSVG-GFP (#11912) is a gift from Dr. Florian Heyd (Freie Universität Berlin, Berlin, Germany). EXT1-YFP and Flag-EXT1 were previously described in Daakour et al. (BMC Cancer 16, 335 (2016)).

Additional cloning vectors used here are: pDEST-mCherry, mEmerald-C1 (Addgene® #53975) and pSYFP2-C1 (Addgene® #22878) or pCS2 EIF ires GFP. The lentiviral constructs used are: shCTRL (anti-eGFP, SHC005, Sigma-Aldrich®) or pLV U6 shRNA NT PGK GFP-T2A-Neo and targeting EXT1 (sh438: TRCN0000039993, sh442: TRCN0000039997, Sigma-Aldrich®). The shRNAs targeting EXT2, EXTL1, EXTL2, and EXTL3 were designed using Vector Builder online platform (https://en.vectorbuilder.com/) and cloned into lentiviral vector pLV-PURO-U6.

Nucleic acids encoding shRNAs used herein are depicted in Table 1 below:

TABLE 1

Nucleic acids encoding shRNAs used herein

| | | |
|---|---|---|
| Name | Sequence | SEQ ID NO: |
| shEXT1-1 | CCGGAGAGCCAGATTGTGCCAACTACTCGAGTAGTTGGCACAATCTGGCTCTTTTTTG | SEQ ID NO: 1 |
| shEXT1-2 | CCGGCCTTCGTTCCTTGGGATCAATCTCGAGATTGATCCCAAGGAACGAAGGTTTTTG | SEQ ID NO: 2 |
| shEXT1-3 | AGCAGACACAATTCTTGTGGGAGGCTTATTTTTCTTCAGTT | SEQ ID NO: 3 |
| shEXT1-4 | ATTACAGATTCCTTCTACAATCAGGTCTATTCATCAGGATA | SEQ ID NO: 4 |
| shEXT1-5 | AAACTTCCGACCCAACTTTGATGTTTCTATTCCCCTCTTTT | SEQ ID NO: 5 |
| shEXT1-6 | AGACAACACCGAGTATGAGAAGTATGATTATCGGGAAATGC | SEQ ID NO: 6 |
| shEXT1-7 | CTCTGCGCCCCTTCGTTCCTTGGGATCAATTGGAAAACGAG | SEQ ID NO: 7 |
| shEXT1-8 | TCATCAGCAGAGCCAGATTGTGCCAACTATCCAAAAACTTA | SEQ ID NO: 8 |
| shEXT1-9 | GCTCTGCGCCCCTTCGTTCCTTGGGATCAATTGGAAAACGA | SEQ ID NO: 9 |
| shEXT1-10 | ACTCATCAGCAGAGCCAGATTGTGCCAACTATCCAAAAACT | SEQ ID NO: 10 |
| shEXT1-11 | TAAAATCCTAGCACTTAGACAGCAGACACAATTCTTGTGGG | SEQ ID NO: 11 |
| shEXT1-12 | CAGCCGGAGAGAAGAACACAGCGGTAGGAATGGCTTGCACC | SEQ ID NO: 12 |
| shEXT1-13 | ACCAATTGGCCAATTGTGAGGACATTCTCATGAACTTCCTG | SEQ ID NO: 13 |
| shEXT1-14 | CGCATGGAGTCCTGCTTCGATTTCACCCTTTGCAAGAAAAA | SEQ ID NO: 14 |
| shEXT1-15 | CCGGCCCAACTTTGATGTTTCTATTCTCGAGAATAGAAACATCAAAGTTGGGTTTTTG | SEQ ID NO: 15 |
| shEXT1-17 | CCGGGCACTTAGACAGCAGACACAACTCGAGTTGTGTCTGCTGTCTAAGTGCTTTTTG | SEQ ID NO: 16 |
| shEXT1-18 | CCGGCCTGCTTCGATTTCACCCTTTCTCGAGAAAGGGTGAAATCGAAGCAGGTTTTTG | SEQ ID NO: 17 |
| shEXT1-20 | CCGGCAAGACTAGGTTGGTACAGTTCTCGAGAACTGTACCAACCTAGTCTTGTTTTTG | SEQ ID NO: 18 |
| shEXT1-21 | CCGGAAGAACACAGCGGTAGGAATCTCGAGATTCCTACCGCTGTGTTCTTCTTTTTG | SEQ ID NO: 19 |
| shEXT1-22 | CCGGCAATTGTGAGGACATTCTCATCTCGAGATGAGAATGTCCTCACAATTGTTTTTG | SEQ ID NO: 20 |
| shEXT1-23 | CCGGATTCTTGTGGGAGGCTTATTTCTCGAGAAATAAGCCTCCCACAAGAATTTTTTG | SEQ ID NO: 21 |
| shEXT1-24 | CCGGAGCCAGATTGTGCCAACTATCCTCGAGGATAGTTGGCACAATCTGGCTTTTTTG | SEQ ID NO: 22 |
| shEXT1-25 | CCGGCTTCGTTCCTTGGGATCAATTCTCGAGAATTGATCCCAAGGAACGAAGTTTTTG | SEQ ID NO: 23 |
| shEXT1-27 | CCGGGAGTATGAGAAGTATGATTATCTCGAGATAATCATACTTCTCATACTCTTTTTG | SEQ ID NO: 24 |

mCherry-RTN4a, mCheryy-ATL1, Lnp1-mCherry lentiviral constructs were a gift from Dr. Tom Rapoport (Dept of Cell Biology, Harvard Medical School, MA, USA). LV-PA-KDEL-GFP is a gift from Dr. Vicky C Jones (University of Central Lancashire, Preston, UK), Lenti-ATL3-GFP is a gift from Dr. Vincent Timmerman (University of Antwerp, Antwerp, Belgium). Lentivirus production and instructions on its use were kindly provided by Viral Vectors core facility (Viral Vectors platform, University of Liege).

### 1.2- Mammalian Cell Lines Generation and Culture

All cell lines HeLa, HEK293, Jurkat, and Cos7 were cultured as previously described in Daakour et al. (see above) and in Hu et al. (Cell 138, 549-561 (2009)). All stable cell lines were generated by lentiviral transduction. Briefly, HEK293T Lenti-x 1B4 cells (Clontech®-Lenti-x HEK293T cells) were transfected with calcium phosphate with three plasmids: the vector of interest, pVSV-G (PT3343-5, Clontech®) and psPAX2 (#12260, Addgene®). The supernatants containing the second-generation viral vectors were harvested and concentrated by ultracentrifugation. The cells (HeLa, HEK293, Jurkat, Cos7) were transduced with the viral vector of interest with MOI (50, 80, 100 depending on the production). After 72 h, the cells were selected for puromycin (Invivogen®) for 3-4 days. For fluorescence-protein-tagged constructs, positive cells were selected by flow cytometry sorting. The cells were finally tested for the presence of mycoplasma (MycoAlert Detection Kit, Lonza® LT07-318), and recombinant viral particles (Lentiviral qPCR TitrationKit, abmGood® #LV900).

### 1.3- DNA-siRNA Transfection

DNA was transfected into HeLa and Cos7 with polyethylenimine (PEI 25 K, Polysciences) as previously described in Daakour et al. (see above). For siRNA transfection, Cos7 and HeLa cells were transfected at 40-50% confluence with 2 nmol of siRNA using a classical calcium-phosphate method according to manufacturer’s instructions (ProFection Mammalian Transfection kit, Promega®). The medium was changed 24 h later and cells were collected 48 h post-transfection. When experiments involved both DNA and siRNA transfection, siRNA transfection was performed, and 24 h later cells were transfected with DNA as described previously (Daakour et al.). Cells were collected 24 h later. The following siRNA duplexes were purchased from Eurogentec® (Belgium) and are depicted in Table 2:

TABLE 2

siRNAs used herein

| | | |
|---|---|---|
| Name | Sequence (from 5′ to 3′) | SEQ ID NO: |
| siEXT1(1) | GGAUCAUCCCAGGACAGGA | SEQ ID NO: 25 |
| siEXT1(2) | GGAUUCCAGCGUGCACAUU | SEQ ID NO: 26 |
| siEXT1(3) | GGCUUAUUUUUCUUCAGUU | SEQ ID NO: 27 |
| siCTRL | GGCUGCUUCUAUGAUUAUG | SEQ ID NO: 28 |

### 1.4- RNA Extraction and RT-qPCR

For expression studies, total RNA was extracted from the cell pellet using Nucleospin RNA kit (Macherey-Nagel®) according to the manufacturer’s instructions. Real-time qPCR was performed using LightCycler® 480 SYBR Green I Master (Roche®) and analyzed in triplicate on a LightCycler (Roche®). The relative expression levels were calculated for each gene using the ΔΔCt method with GAPDH as an internal control. Primer sequences for qPCR are depicted in Table 3 below:

TABLE 3

primers used herein

| | | |
|---|---|---|
| Name | Sequence (from 5′ to 3′) | SEQ ID NO: |
| EXT1 Forward | GCTCTTGTCTCGCCCTTTTGT | SEQ ID NO: 29 |
| EXT1 Reverse | TGGTGCAAGCCATTCCTACC | SEQ ID NO: 30 |
| GAPDH Forward | TTGCCATCAATGACCCCTTCA | SEQ ID NO: 31 |
| GAPDH Reverse | CGCCCCACTTGATTTTGGA | SEQ ID NO: 32 |

### 1.5- Immunofluorescence and Confocal, Super-Resolution Microscopy

3×10⁴ Cos7 and 5×10⁴ HeLa cells were grown on 18 mm round glass coverslips and transfected with 500 ng of DNA/well. For immunostaining, the cells were washed with PBS (pH 7.4) and fixed with 4% paraformaldehyde in PBS for 15 min at RT. Cells were permeabilized with 0.5% Triton X-100 for 10 min and incubated with blocking solution (0.025% Tween-20 and 10% FBS) for 30 min. Primary antibody staining was performed overnight at 4° C. in 5% blocking solution: mouse-anti-betacatenin 1:1,000 (Santa Cruz®), mouse-anti-Calnexin 1:500 (Abcam®), rabbit-anti-EXT1 1:100 (Prestige Antibodies Sigma-Aldrich®), mouse-anti-HS (10E4) (1:100, USBio®), rabbit-anti-GM130 1:3,200 (Cell Signaling®), mouse-anti-PDIA3 1:1,000 (Prestige Antibodies Sigma-Aldrich®), mouse-anti-SEC31 1:500 (BD Bioscience®). Goat-antirabbit, donkey-anti-rabbit or goat-anti-mouse secondary antibodies labeled with Alexa Fluor 488 or Texas Red (ThermoFisher Scientific®), anti-mouse-STAR-Red (Abberior®) were used at a 1:2,000 dilution for 1 h. Cells were stained with DAPI (Thermo Fisher Scientific®) when needed for 5 min at RT, washed 5 times with PBS and mounted with Prolong Antifade Mountants (Thermo Fisher Scientific®). Slides were analyzed by confocal microscopy with a Leica TCS SP8 microscope using the 100× oil objective. Images were taken at 2068×2068 pixel resolution and deconvoluted with Huygens Professional software. SYFP2-EXT1 was analyzed by Stimulated Emission Depletion (STED) microscopy with a Leica SP8 STED 592 nm laser. Images were taken at 2068×2068 pixel resolution and deconvoluted with Huygens Professional software. SEC31 was analyzed with Stedycon STED laser 775 nm. mEmerald-EXT1 was analyzed by Structured Illumination Microscopy (SIM) super-resolution. SIM imaging was performed at the Cell Imaging and Cytometry Core facility (Turku University) using a DeltaVision OMX SR V4 microscope using a 60x/1.42 Olympus Plan Apo N SIM objective and sCMOS cameras (Applied Precision®), 2560×2160 pixel resolution. The SIM image reconstruction was performed with DeltaVision softWoRf 7.0 software. For live imaging of Cos7 cells expressing mCherry-ATL1 or Lnp1-mCherry, 3×10⁴ cells were plated and imaged at 37° C. and 5% CO₂ in a thermostat-controlled chamber on a Zeiss LSM800 AiryScan Elyra S1 SR confocal microscope using the 63× oil objective at 1 frame/100 ms for 5 s. Further analysis was performed in ImageJ software.

### 1.6- Image Analysis

For colocalization analysis, the average Pearson’s correlation coefficient test was performed with the plugin Colocalization Threshold in ImageJ software. To track the displacement of main junctions during successive frames, the dynamic features of the cell were retrieved from the time-lapses of Cos7 cells expressing mCherry-ATL1 or Lnp1-mCherry with the following image processing procedure. Images were preprocessed to uniformize the intensities. Then, each image was binarized and skeletonized using Matlab2016a. The skeleton was labeled using AnalyzeSkeleton plugin from ImageJ. From this process, each pixel of the skeleton was classified according to its neighborhood leading to three-pixel classes: end-point, junctions and tubules. To reflect the structure of the ER, the ratio of the junctions over the tubules was computed for mCherry-ATL1 and Lnp1-mCherry proteins. The dynamics of the ER was assessed by the main junctions displacement during a timelapse. To achieve the tracking of the displacement, the junctions larger than three pixels were kept segmented. Then, the segmented objects were multiplied by the initial image intensity to consider the initial light intensity. Finally, a gaussian blur was applied to these objects. The tracking of the bright spot was achieved by using a single-particles tracking algorithm, the “simple LAP tracker” available in ImageJ plugin TrackMate. The parameters were set following the recommendations for Brownian motion like’s movements, i.e., a max linking distance of seven pixels, a max closing distance of ten pixels and a max frame gap of three pixels. From the results of Trackmate, only the tracks longer than ten frames were kept in order to reduce the noise. Finally, using all velocity vectors measured, a cumulative velocity distribution was computed. Furthermore, a diffusion coefficient based on instantaneous velocity was computed using the Matlab as described previously in Holcman et al. (Nat. Cell Biol. 20, 1118-1125 (2018)). In AnalyzER, original images were imported, and the regions of interest segmented using Otsu’s method (Threshold Selection Method from Gray-Level Histograms. IEEE Trans. Syst. Man. Cybern. 9, 62-66 (1979)). Cisternae are identified using an image opening function and active contour refinement. The tubular network is enhanced using phase congruency, and the resulting enhanced network is skeletonized to produce a single-pixel wide skeleton running along each tubule. Regions fully enclosed by the skeletonized tubular network and the cisternae are defined as polygonal regions, and features such as area, circularity, and elongations are extracted.

### 1.7- Photoactivatable GFP Imaging

Using an adaptation of a published assay (Krols et al. Cell Rep. 23, 2026-2038 (2018)), 3×10⁴ Cos7 cells expressing PA-GFP-KDEL were plated, and live imaging was performed at 37° C. and 5% CO₂ in a thermostat-controlled chamber on a Zeiss LSM800 AiryScan Elyra S1 SRconfocal microscope using the 100× oil-objective. PA-GFP-KDEL was activated at a perinuclear ER region using the 405 nm laser at 100%, after which the cell was imaged at 1 frame/500 ms for 90 s using the 488 nm laser. Fluorescence intensities were measured using ImageJ software, and data analysis and curve fitting were performed in Graphpad Prism 8 (Graphpad Software). To avoid inter-cell variability, the activation site was at the perinuclear area of cells with the same ER density. The integrated fluorescence intensity of each region of interest (ROI) at fixed distances (8, 12, 16 µm) from the activation region was measured in ImageJ. Normalization of raw values was done, by defining the initial fluorescence to zero and the maximum fluorescence to 1 for each ROI. Image analysis was performed in ImageJ.

### 1.8- Rush Assay

HeLa cells were transfected with Str-KDEL-TNF-SBP-mCherry construct as described above, and 24 h after transfection mCherry positive cells were sorted. 5 × 10⁴ cells were cultured on 35 mm imaging dish. The day after, cells were transferred at 37° C. in a thermostat-controlled chamber. At time point zero, the medium was removed and replaced with medium containing D-biotin (Sigma-Aldrich) at 40 µM concentration. The timelapse acquisition was made using a Zeiss LSM800 AiryScan Elyra S1 SR confocal microscope. Images were acquired using a 63× oil-objective. For each time point, the integrated intensity of a region of interest (ROI) was measured. The integrated intensity of an identical size ROI corresponding to background was measured and subtracted from the values of the integrated intensity for each time point. The values were then normalized to the maximum value. These quantifications were performed using the Zeiss Black software.

### 1.9- Export Assay

3 ×10⁴ Cos7 cells were cultured on 35 mm imaging dish, and transfected with the ts045-VSVG-GFP reporter construct and immediately incubated at 40° C. overnight to retain the reporter protein in the ER. After the addition of cycloheximide, cells were transferred in a thermostat-controlled chamber at 40° C. The temperature was shifted to 32° C., and cells were processed for immunofluorescence at t=0, t=45 and t=90 min and stained with mouse-anti-beta-catenin antibody as described above. The acquisition was made using a Zeiss LSM800 AiryScan Elyra S1 SR confocal microscope. Images were acquired using a 40x oil-objective.

### 1.10- Calcium Flux Detection Assay

2×10⁵ Cos7 cells were washed twice and processed for immunofluorescence. Fluo-4, AM Loading Solution was added on the cells according to manufacturer’s instructions (Fluo-4 Calcium Imaging Kit, Thermo Fisher Scientific®). Images were acquired using a Leica TCS SP5 confocal microscope and the 63× oil objective; the analysis was performed in ImageJ software.

### 1.11- Transmission Electron Microscopy

HeLa, HEK293 and Jurkat shCTRL (Sigma cat#SHC005) and shEXT1 (shEXT1-1), were fixed for 90 min at 4° C. with 2.5% glutaraldehyde in Sorensen 0.1 M phosphate buffer (pH 7.4), and post-fixed for 30 min with 2% osmium tetroxide. Following, dehydration in graded ethanol, samples were embedded in Epon. Ultrathin sections obtained with a Reichert Ultracut S ultramicrotome were contrasted with uranyl acetate and lead citrate. The analysis was performed with a JEOL JEM-1400 transmission electron microscope at 80 kV and in a Tecnai Spirit T12 at 120 kV (Thermo Fisher Scientific®).

### 1.12- Immunohistochemistry

Immunohistochemical experiments were performed using a standard protocol previously described in Hubert et al. (J. Pathol. 234, 464-77 (2014)). In the present study, the antigen retrieval step was: citrate pH 6.0 and the following primary antibody was used: anti-EXT1 (⅟50, ab 126305, Abcam®). The rabbit Envision kit (Dako®) was used for the secondary reaction.

### 1.13- Preparation of Microsomes From Cultured Cells

HeLa cells expressing FLAG-EXT1 or HeLa shCTRL and shEXT1 (2×10⁸) were harvested and washed with PBS and with a hypotonic extraction buffer (10 mM HEPES, pH 7.8, with 1 mM EGTA and 25 mM potassium chloride) supplemented with a protease inhibitors cocktail. Cells were resuspended in an isotonic extraction buffer (10 mM HEPES, pH 7.8, with 0.25 M sucrose, 1 mM EGTA, and 25 mM potassium chloride) supplemented with a protease inhibitors cocktail and homogenized with 10 strokes using a Dounce homogenizer. The suspension was centrifuged at 1,000×g for 10 min at 4° C. The supernatant was centrifuged at 12,000×g for 15 min at 4° C. The following supernatant fraction, which is the post mitochondrial fraction (PMF), is the source for microsomes. The PMF was centrifuged for 60 min at 100,000×g at 4° C. The pellet was resuspended in isotonic extraction buffer supplemented with a protease inhibitors cocktail and stored in -80° C. Isolated membranes were boiled 5 min in 2× SDS-loading buffer. Then, solubilized samples were separated on SDS-PAGE and analyzed by western blotting.

### 1.14- Western Blotting and Antibodies

Cells were lysed in immunoprecipitation low salt buffer (IPLS: 25 mM Tris-HCl pH 7.4, 150 mM NaCl, 1 mM EDTA, 1% NP-40 and 5% glycerol, complete Protease Inhibitor (Roche®) and Halt Phosphatase Inhibitors (Thermo Fisher Scientific®)). Concentrations were determined using the Bradford assay. SDS-PAGE and western blotting were performed using standard protocols. The following primary antibodies were used: mouse-anti-Calnexin 1:2,000 (Abeam®), rabbit-anti-EXT1 1:500 (Prestige Antibodies, Sigma-Aldrich®), mouse-anti-NogoA (Santa Cruz®), rabbit-anti-FLAG 1:4,000 (Sigma-Aldrich®), mouse-anti-FLAG 1:4,000 (Sigma-Aldrich®), goat-anti-actin 1:2,000 (Santa Cruz®), rabbit-anti-HSP70 1:3,000 (Santa Cruz®). Dad1, STT3b, STT3a, Sec61A, Trap-alpha, TRAP-beta, SEC62, SEC63 were a kind gift from Dr. Richard Zimmermann (Medical Biochemistry and Molecular Biology, Saarland University, Homburg, Germany). The following conjugated secondary antibodies were used: a-mouse-HRP 1:5,000 (Santa Cruz®), a-rabbit-HRP 1:5,000 (Santa-Cruz®), anti-goat 1:5,000 (Santa-Cruz®).

### 1.15- Affinity Purification for Mass Spectrometry

2× solubilization buffer (3.5% digitonin, 100 mM HEPES (pH 7.5), 800 mM KOAc, 20 mM MgOAc2, 2 mM DTT) was mixed in a ratio 1:1 with the microsomal fraction and incubated 10 min on ice. Samples were centrifuged for 15 min at 14,000 rpm to isolate the solubilized material and remove the insoluble material. The supernatant was further used for immunoprecipitation. Equilibrated agarose beads M2-FLAG (Sigma-Aldrich®) were added in the microsomal fraction (15 µl of beads per half of a 10-cm cell culture dish), and rotation was performed overnight at 4° C. Beads were washed 3 times for 15 min with glycine 50 mM pH 3.0 for protein elution. The supernatant was supplemented with Tris-HCL pH 8.0. Eluted proteins were then subjected to trypsin digestion and identified by mass spectrometry. Mass spectrometry analyses were performed by the GIGA-Proteomics facility, University of Liege or the proteomic core facility of de Duve Institute, Brussels, Universite Catholique de Louvain, Belgium. As a control, beads were washed five times with IPLS and eluted by boiling 5 min in 2× SDS-loading buffer. Then, solubilized samples were separated on SDS-PAGE and analyzed by western blotting.

### 1.16- Mass Spectrometry

Peptides were dissolved in solvent A (0.1% TFA in 2% ACN), directly loaded onto reversed-phase pre-column (Acclaim PepMap 100, Thermo Fisher Scientific®). Peptide separation was performed over 140 min using a reversed-phase analytical column (Acclaim PepMap RSLC, 0.075×250 mm, Thermo Fisher Scientific®) with a linear gradient of 4%-32% solvent B (0.1% FA in 98% ACN) for 100 min, 32%-60% solvent B for 10 min, 60%-95% solvent B for 1 min and holding at 95% for the last 6 min at a constant flow rate of 300 nl/min on an Ultimate 3000 UPLC system. The resulting peptides were analyzed by Orbitrap Fusion Lumos tribrid mass spectrometer using a high-low data-dependent scan routine for protein identification and an acquisition strategy termed HCD product-dependent EThcD/CID (Thermo Fisher Scientific®) for glycopeptides analysis.

Briefly for the latter, the peptides were subjected to NSI source and were detected in the Orbitrap at a resolution of 120,000. Peptides were selected for MS/MS using HCD setting as 28 and detected in the Orbitrap at a resolution of 30,000. If predefined glycan oxonium ions were detected in the low m/z region it triggered an automated EThcD and CID spectra on the glycopeptide precursors in the Orbitrap. A data-dependent procedure that alternated between one MS scan every 3 seconds and MS/MS scans was applied for the top precursor ions above a threshold ion count of 2.5^{E4} in the MS survey scan with 30. 0 s dynamic exclusion. MS1 spectra were obtained with an AGC target of 4^{E5} ions and a maximum injection time of 50 ms, and MS2 spectra were acquired in the Orbitrap at a resolution of 30.000 with an AGC target of 5^{E4} ions and a maximum injection time of 300 ms. For MS scans, the m/z scan range was 350 to 1,800. For glycopeptide identification the resulting MS/MS data was processed using Byonic 3.5 (Protein Metrics®) search engine within Proteome Discoverer 2.3 against a human database obtained from Uniprot, the glycan database was set to “N-glycan 182 human no multiple fucose or O-glycan 70 human”. Trypsin was specified as cleavage enzyme allowing up to 2 missed cleavages, 5 modifications per peptide and up to 7 charges. Mass error was set to 10 ppm for precursor ions and 20 ppm for fragment ions. Oxidation on Met, carbamidomethyl (+57.021 Da) were considered as variable modifications on Cys. Glycopeptides with a Byoinic score >= 300 and with a Log Prob >= 4.0 were retained and their identification was manually validated.

### 1.17- SILAC Labeling

HeLa cells (shCTRL, shEXT1) were cultured for at least five cell doublings in either isotopically light or heavy SILAC DMEM obtained from Thermo Scientific® (catalog number A33969) containing 10% FBS and 50 µg/ml streptomycin and 50 units/ml penicillin (Lonza®). For the heavy SILAC medium, 50 mg of 13C6 L-Lysine-2HCl (heavy) and 50 mg of L-Arginine-HCl was added. In light SILAC medium 50 mg of LLysine-2HCl (light) and 50 mg of L-Arginine-HCl was added. 2×10⁵ cells adapted to grow in DMEM. The cell pellet was suspended in 150 µL of modified RIPA buffer and sonicated followed by incubation at 60° C. for 15 min. Samples were clarified by centrifugation; each replicate was pooled and quantified by Qubit (Invitrogen®): 20 µg of the sample was separated on a 4-12% Bis-Tris Novex mini-gel (Invitrogen®) using the MOPS buffer system. The gel was stained with Coomassie, and gel bands were excised at 50 kDa and 100 kDa. Gel pieces were processed using a robot (ProGest, DigiLab). They were washed with 25 mM ammonium bicarbonate followed by acetonitrile and reduced with 10 mM dithiothreitol at 60° C. followed by alkylation with 50 mM iodoacetamide at RT and digested with trypsin at 37° C. for 4 h. Finally, they were quenched with formic acid, and the supernatant was analyzed directly without further processing. For the SILAC analysis performed by MS Bioworks LLC (MI, USA), the samples were pooled 1:1 and 20 µg was separated on a 4-12% Bis-Tris Novex minigel (Invitrogen®) using the MOPS buffer system. The gel was stained with Coomassie, and the lanes excised into 40 equal segments using a grid. For mass spectrometry, the gel digests were analyzed by nano-LC/MS/MS with a Waters NanoAcquity HPLC system interfaced to a Thermo Fisher Q Exactive. Peptides were loaded on a trapping column and eluted over a 75 µm analytical column at 350 nL/min. Both columns were packed with Luna C18 resin (Phenomenex®). The mass spectrometer was operated in data-dependent mode, with MS and MS/MS performed in the Orbitrap at 70,000 FWHM and 17,500 FWHM resolution, respectively. The fifteen most abundant ions were selected for MS/MS. Data were processed through the MaxQuant software 1.5.3.0 (www.maxquant.org) which served several functions such as the recalibration of MS data, the filtering of database search results at the 1% protein and peptide false discovery rate (FDR), the calculation of SILAC heavy: light ratios and data normalization. Data were searched using a local copy of Andromeda with the following parameters, Enzyme set as trypsin, database set as Swissprot Human (concatenated forward and reverse plus common contaminant proteins), fixed modification: Carbamidomethyl (C), variable modifications: Oxidation (M), Acetyl (Protein N-term), 13C6 (K) and fragment Mass Tolerance: 20 ppm.

### 1.18- Metabolomics Profiling

For metabolite quantification, HEK293 shCTRL, and shEXT1 cells were seeded in triplicate (n=3) in 6-well plates with DMEM supplemented with 10% FBS. After 24 h, the media was removed and replaced with fresh media containing stable isotopic tracer 13C-glucose. For one well per condition, the medium was replaced with 1-12Cglucose.

Upon reaching 70% confluency, the supernatant was stored in -80° C. and cells were washed twice with PBS, harvested and the cell pellet stored in -80° C. until Liquid Chromatography/Mass Spectrometry identification of metabolites at the University of Leuven metabolomics core facility.

### 1.19- N-Glycans and O-Slycans Profiling

Microsomes were isolated as described above, and glycans profiling performed by Creative Proteomics (NY, USA). For the preparation of N-glycans ~250 µgof lyophilized protein samples are required. The dry samples are resuspended in fresh 2 mg/ml solution of 1,4-dithiothreitol in 0.6 M TRIS buffer pH 8.5 and incubated at 50° C. for 1 h. Fresh 12 mg/ml solution of iodoacetamide in 0.6 M TRIS buffer pH 8.5 was added to the DTT-treated samples and incubated at RT in the dark for 1 h. Samples were dialyzed against 50 mM ammonium bicarbonate at 4° C. for 16-24 h, changing the buffer 3 times. The molecular cut-off should be between 1 and 5 kDa. After dialysis, the samples were transferred into 15 ml tubes and lyophilized. Following resuspension of the dry samples in 0.5 ml of a 50 µg/ml solution of TPCK-treated trypsin in 50 mM ammonium bicarbonate and overnight incubation at 37° C. The reactions stopped by adding 2 drops of 5% acetic acid. Condition a C18 Spe-Pak (50 mg) column with methanol, 5% acetic acid, 1-propanol and 5% acetic acid. Trypsin-digested samples were loaded onto the C18 column and then column was washed with 4 ml of 5% acetic acid and the peptides eluted from the C18 column with 2 ml of 20% 1-propanol, then 2 ml 40% 1-propanol, and finally 2 ml of 100% isopropanol. All the eluted fractions were pooled and lyophilized. The dried material was resuspended thoughtfully in 200 µl of 50 mM ammonium bicarbonate and 2 µl of PNGaseF was added, following incubation at 37° C. for 4 h. Then, another 3 µl of PNGaseF was added for overnight incubation at 37° C. To stop the reaction addition of 2 drops of 5% acetic acid is required. Condition a C18 Spe-Pak (50 mg) column with methanol, 5% acetic acid, isopropanol and 5% acetic acid and the PNGaseF-digested samples were loaded onto the C18 column, and flow-through was collected. The column was washed with 4 ml of 5% acetic acid, and fractions were collected. Flow-through and wash fractions were pooled, samples were lyophilized and proceeded to permethylation.

For the O-glycans preparation, 1 ml of 0.1 M NaOH was added to 55 mg of NaBH₄ in a clean glass tube and mixed well, and 400 µl of the borohydride solution was added to the lyophilized sample (collected peptides/glycopeptides after PNGaseF digestion). Following, incubation at 45° C. overnight, the reaction was terminated by the addition of 4-6 drops of pure (100%) acetic acid, until fizzing stops. A stock solution of Dowex 50 W ×8 (mesh size 200-400) was made by washing three times 100 g of resin with 100 ml of 4 M HCl. The resin was washed with 300 ml of Milli-Q water, and the wash step was repeated for ~15 times until the pH remained stable. The resin was then washed with 200 ml of 5% acetic acid three times. A desalting column with 2-3 ml of the Dowex resin prepared above in a small glass column. The column was washed with 10 ml of 5% acetic acid. Acetic acid-neutralized samples were loaded onto the column and washed with 3 ml of 5% acetic acid. Flow-through was pooled and washed. The collected material was lyophilized, supplemented with 1 ml of acetic acid: methanol (1:9; v/v=10%) solution, vortexed thoroughly and dried under a stream of nitrogen. This co-evaporation step was repeated for three more times. Condition a C18 Spe-Pak column with methanol, 5% acetic acid, isopropanol and 5% acetic acid. The dried sample was resuspended in 200 µl of 50% methanol and loaded onto the conditioned C18 column. The column was washed with 4 ml of 5% acetic acid. Flowthrough was collected, pooled, and washed. Lyophilized samples were processed to permethylation.

For the permethylation, the preparation of the slurry NaOH/DMSO solution is made fresh every time. Mortar, pestle, and glass tubes were washed with Milli-Q water and dried beforehand. Whenever possible, liquid reagents were handled with disposable glass pipettes. Solvents are HPLC grade or higher. With a clean and dry mortar and pestle grind 7 pellets of NaOH in 3 ml of DMSO. One ml of this slurry solution was added to a dry sample in a glass tube with a screw cap and supplemented with 500 µl of Iodomethane and incubated at RT for 30 min. The mixture turns white and even becomes solid as it reaches completion. One ml of Milli-Q water was added to stop the reaction, and the tube was vortexed until all solids were dissolved. The sample was supplemented with 1 ml of Chloroform and additional 3 ml of Milli-Q water, vortexed and centrifuged briefly to separate the chloroform and the water phases (~5,000 rpm, <20 sec). The aqueous top layer was discarded and wash 2 more times. Chloroform fraction dried with a SpeedVac (~20-30 min). Condition a C18 Spe-Pak (200 mg) column with methanol, Milli-Q water, and acetonitrile. Dry samples were resuspended in 200 µl of 50% methanol and loaded onto the column. The tube was washed with 1 ml of 15% acetonitrile and loaded onto the column. The column was washed with 2 ml of 15% acetonitrile, then eluted in a clean glass tube with 3 ml of 50% acetonitrile. Lyophilized eluted fraction for MS analysis was used. MS data were acquired on a Bruker UltraFlex II MALDI-TOF Mass Spectrometer instrument. The positive reflective mode was used, and data were recorded between 500 m/z and 6,000 m/z for N-glycans and between 0 m/z and 5,000 m/z for O-glycans. For each MS N- and O-glycan profiles the aggregation of 20,000 laser shots or more were considered for data extraction. Mass signals of a signal/noise ratio of at least 2 were considered and only MS signals matching an N- and O-glycan composition was considered for further analysis and annotated. Subsequent MS post-data acquisition analysis was made using mMass (see Strohalm et al., Anal. Chem. 82, 4648-4651 (2010)).

### 1.20- Glycosyltransferase Assay

Glycosyltransferase activity of microsomes from HeLa shCTRL, and shEXT1 was determined with the Glycosyltransferase Activity Kit (R&D Systems®). A glycosyltransferase reaction was carried out in 50 µL of reaction buffer in a 96-well plate at room temperature for 20 min, according to the manufacturer’s instructions. The absorbance value for each well was measured at 620 nm with a microplate reader TECAN Infinite®200 PRO.

### 1.21- RNA Sequencing

RNA sequencing analysis was previously described in Daakour et al. (see above). Model generation and flux balance analysis Model generation and in silico flux balance analysis was done using the Constraint-Based Reconstruction Analysis (COBRA) toolbox V3.0 in the Matlab 2018a environment with an interface to IBM Cplex and GLPK solvers provided in the COBRA toolbox. Linear programing problems were solved on a macOS Sierra version 10.12.6. To generate the control and EXT1 knocked down specific models, the gene expression mRNA data for samples of control EXT1 knocked down cells (RNA seq) were integrated with the COBRA human model, RECON2. The integration step uses the GIMME algorithm, available in the COBRA toolbox. Because GIMME requires binary entries for the indication of the presence or absence of genes, we used a gene expression threshold value equals to the first quartile RPKM (reads per kilobase of transcript per million) for genes in control and EXT1 knocked-down cells. GIMME only integrates reactions associated with active genes, leaving those associated with the lowly expressed genes inactive. Therefore, genes with expression values below the threshold were given the value of 0 (inactive), and those with expression values higher than the threshold were given a value of 1 (active). Flux balance analysis (FBA) calculates the flow of metabolites through a metabolic network, thereby predicting the flux of each reaction contributing to an optimized biological objective function such as growth rate. Simulating growth rate requires the inclusion of a reaction that represents the production of biomass, which corresponds to the rate at which metabolic precursors are converted into biomass components, such as lipids, nucleic acids, and proteins. For both models generated after the integration step, we used the biomass objective function as defined in the RECON2 model to obtain the FBA solution using the COBRA Toolbox command, optimizeCbModel. After identification of the objective function in the model, the entries to the command optimizeCbModel are: the model and the required optimization of the objective function (maximum production). The command output is the FBA solution, which includes the value of the maximum production rate of the biomass and a column vector for the conversion rate value (reaction fluxes) of each metabolite accounted for in the model.

### 1.22- Statistical Analysis

Graph values are represented as mean + s.d. (standard deviation) of the mean calculated on at least three independent experiments/samples. The analyses were performed in Prism 8 (Graphpad Software). The statistical significance between means was determined using one-way ANOVA followed by two-tailed, unpaired Student’s t-test. p-values thresholds depicted as follows: *p<0.05, **p<0.01; ***p<0.001; ****p<0.0001; n.s., not significant. Significance for PA-GFP-KDEL was performed using two-way ANOVA followed by Sidak’s multiple comparisons test. Significance for Rush assay was performed using the two-stage linear step-up procedure of Benjamini, Krieger and Yekutieli, with Q=1%. Each time point was analyzed individually, without assuming a consistent SD.

### 2- Results

### 2.1- EXT1 Subcellular Localization in ER Tubules and Sheet Matrices

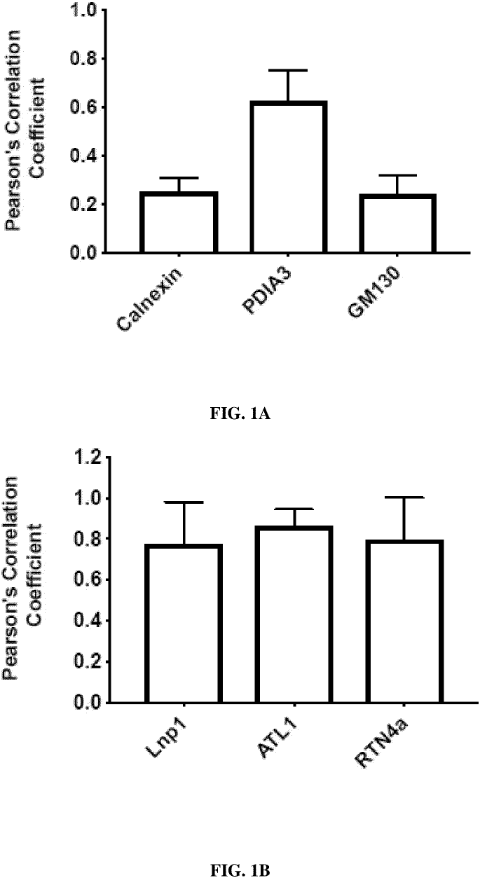

Using conventional confocal microscopy, previous studies have shown that overexpressed EXT1 localizes predominantly to the ER. To overcome spatial limitations of optical microscopy and precisely characterize EXT1 localization in ER structures, super-resolution imaging (SR) was used. EXT1 construct tagged with SYFP2 and mEmerald, two fluorophores with different photostability properties were transiently expressed in Cos7 cells. Using two SR technologies, Stimulated Emission Depletion (STED) and Structured Illumination Microscopy (SIM) it was observed that EXT1 localized in dense sheets and the peripheral ER tubules. EXT1 largely co-localized with the ER luminal marker protein disulfide isomerase family A member 3 (PDIA3) and to a lesser extent with lectin chaperone calnexin and Golgi marker GM130 (FIG. 1A). Also, EXT1 almost perfectly colocalized with ER-shaping proteins Lunaparkl (Lnpl), ATL1 and RTN4a in tubules and the ER three-way junctions (FIG. 1B), further confirming the localization of EXT1 in ER structures.

### 2.2- EXT1 Depletion Affects ER Morphology and Luminal Dynamics

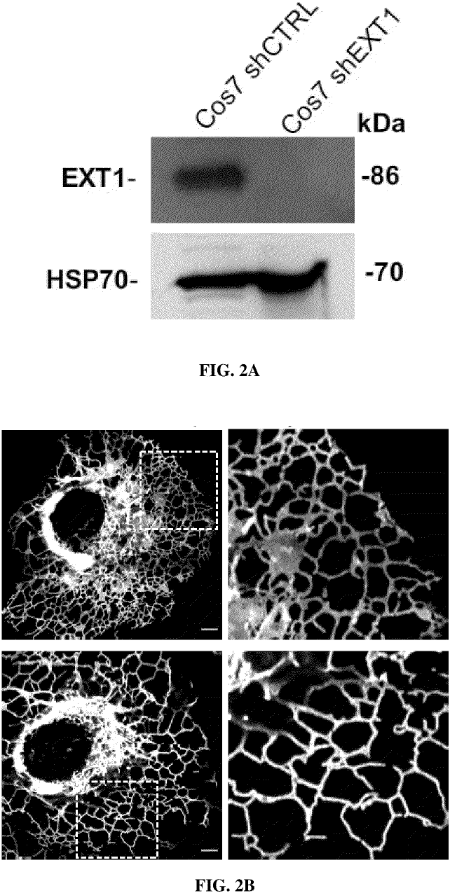

The ER morphology was significantly altered in Cos7 KD_EXT1 (knockdown of EXT1) cells where it appeared asymmetrically dispersed in its periphery in comparison with control cells (FIGS. 2A-D).

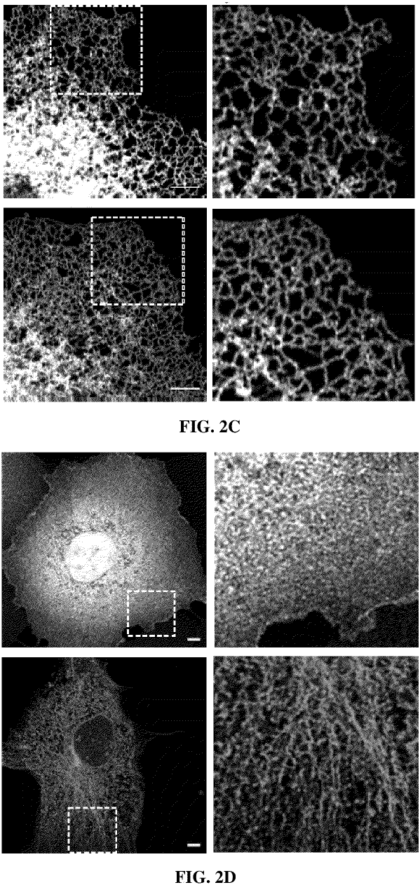

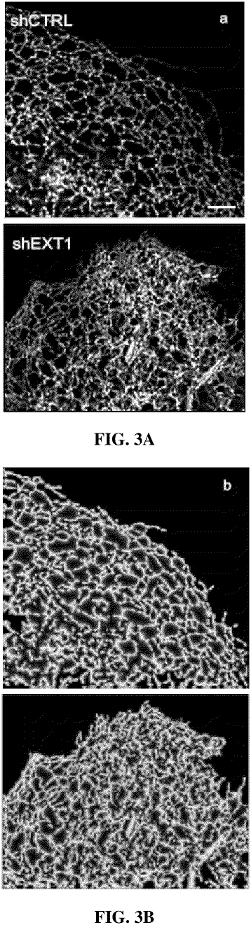

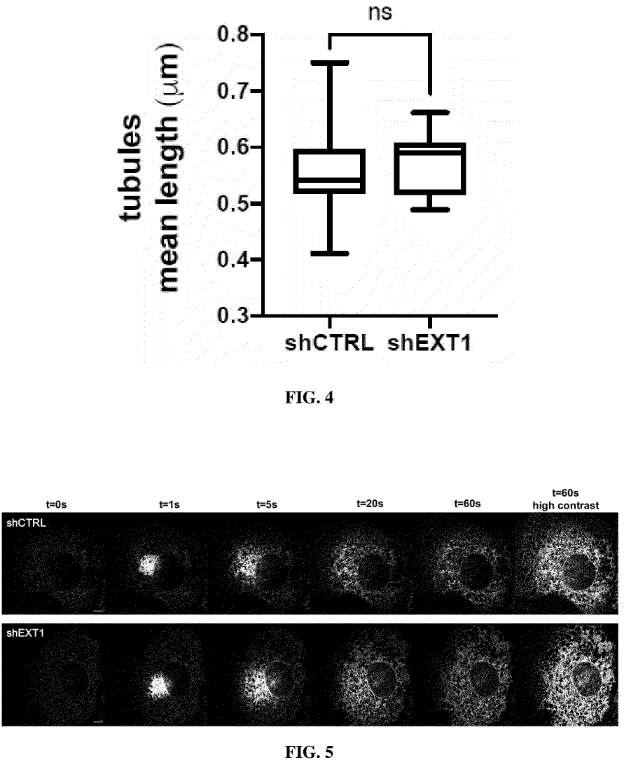

To analyze the ER luminal structural rearrangements, ER membrane structures marked with SEC61b were quantified by using a segmentation algorithm that excludes insufficient fluorescent intensity to give a single-pixel-wide network and allows quantification of individual tubule morphological features. ER cisternae were detected independently using the image opening function followed by active contour refinement. The tubular ER network was altered in KD _EXT1 cells and exhibited a denser reticulated phenotype in comparison to control (FIGS. 3A-B). Measurements of the polygonal area of ER tubular network confirmed our observations with a reduction from 0.946 µm² in control to 0.778 µm² in KD_EXT1 condition. Other tubular and cisternal ER metrics (such as, e.g., tubules mean length), cisternae mean area, perimeter mean length) remained unaffected (FIG. 4), suggesting that the denser tubular network might indicate a more crowded ER lumen in KD_EXT1 cells. Accordingly, the molecular chaperone calnexin, which assists protein folding in the ER, exhibited an aggregation pattern in KD_EXT1 cells. This aggregation might result in a decreased movement of molecules through the ER lumen. To assess how a reduced polygonal area following EXT1 knockdown might influence ER luminal protein mobility and network continuity, the relative diffusion and active transport through the ER lumen of a photoactivable ER lumen marker (PA-GFP-KDEL) was quantified. It was observed that PA-GFP-KDEL was spread throughout the entire ER network, suggesting that the continuity of ER was not affected in cells knocked down for EXT1. However, in KD_EXT1 cells, it was observed a significantly higher dynamic of fluorescence intensity in regions closer to the nucleus (FIG. 5) suggesting that the structural rearrangements of the ER following EXT1 knockdown actively participate in luminal protein transport. Altogether, these data demonstrate that EXT1 induces ER morphological changes that impair protein movement through the ER.

### 2.3- EXT1 Knockdown Results in Increased Secretory Cargo Trafficking

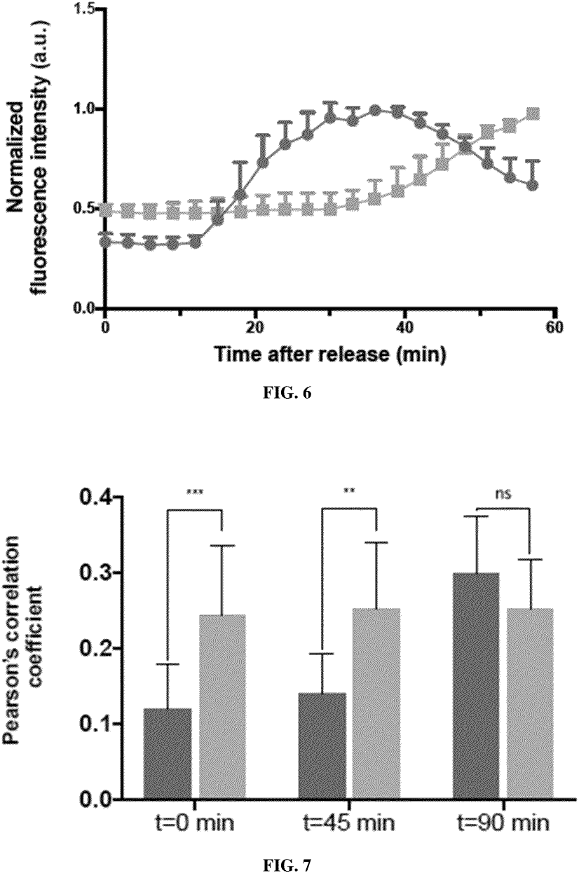

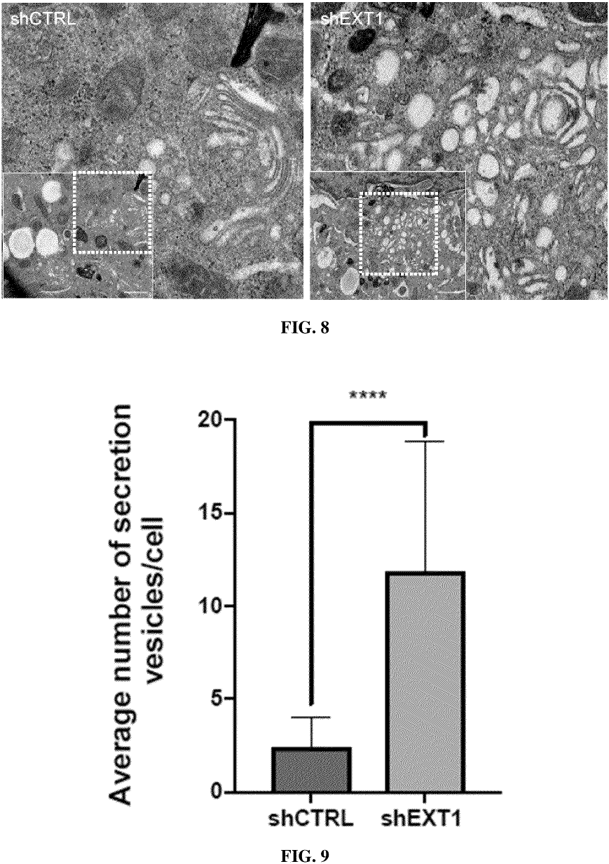

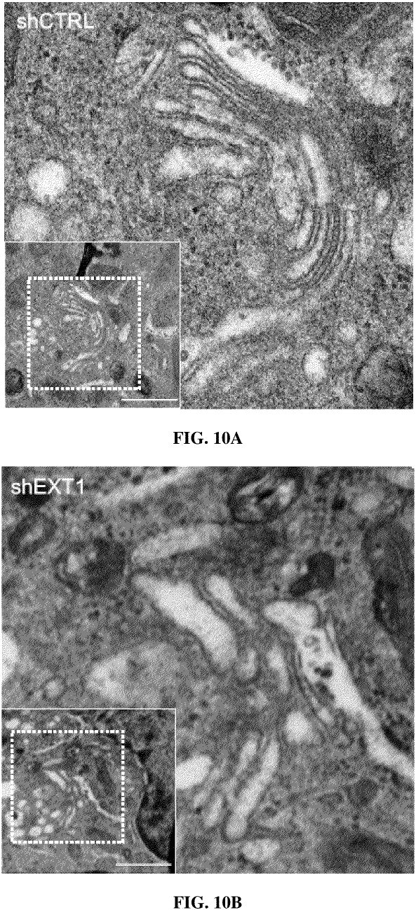

To comprehensively assess the function of EXT1 in the ER, interactome analysis was combined with imaging approaches. First, the EXT1 interactome in ER microsomes was captured by affinity purification and mass spectrometry analysis. Consistent with a role in ER morphology, spatial analysis of functional enrichment (SAFE) analysis identified three functional modules within EXT1 interactors, two of which being translation initiation and protein targeting to the ER. Next, potential connections between EXT1 and the secretory pathway were investigated by comparing the proteome isolated from control and KD_EXT1 cells after stable isotope labeling by amino acids (SILAC). To further assess the changes in the secretory pathway, anterograde transport was monitored using the retention selective hook (RUSH) system that enables the synchronization of cargo trafficking. By tracking cargo transport from the ER to the Golgi using live imaging, it was observed a slower dynamic response in KD_EXT1 cells resulting in an increased residency of the cargo within the secretory pathway (FIG. 6). This finding was confirmed using an additional ER export assay based on the vesicular-stomatitis-virus glycoprotein (VSVG) (FIG. 7), and by examining COPII coat structural components SEC16 and SEC31. Finally, transmission electron microscopy (TEM) indicated a higher number of trans-Golgi secretory vesicles (2.41±1.58 and 11.83±7.00 secretion vesicles/cell, shCTRL, and shEXT1, respectively) following depletion of EXT1 in HeLa cells (FIG. 8 and FIG. 9). Altogether, these observations demonstrate that the EXT1 structural role in the ER correlates with functional consequences on secretion.

### 2.4- EXT1 Depletion Induces ER Extension and Golgi Re-Organization

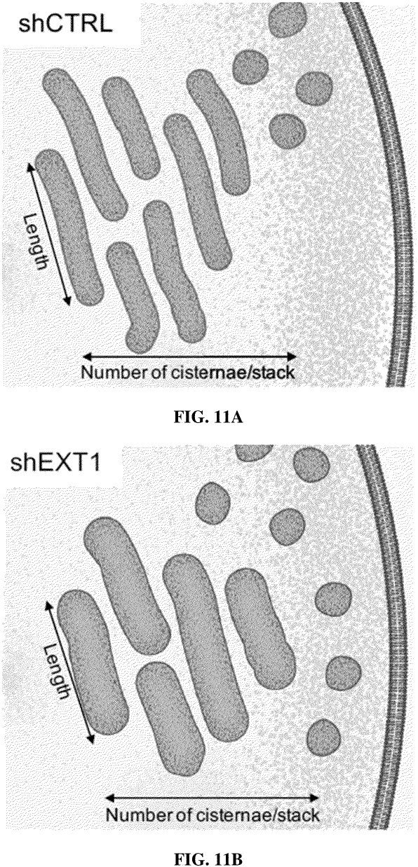

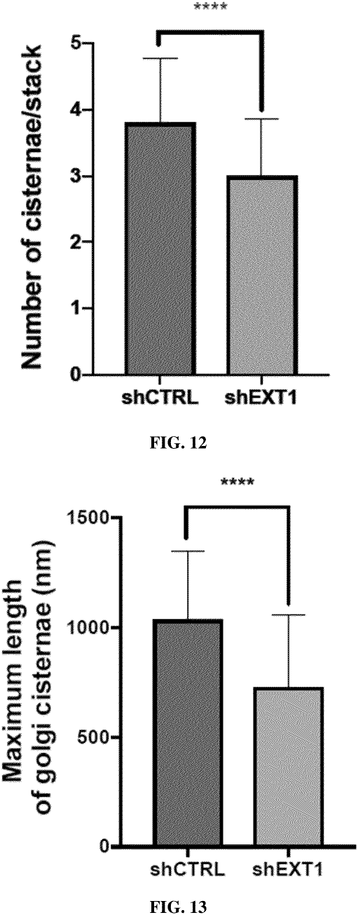

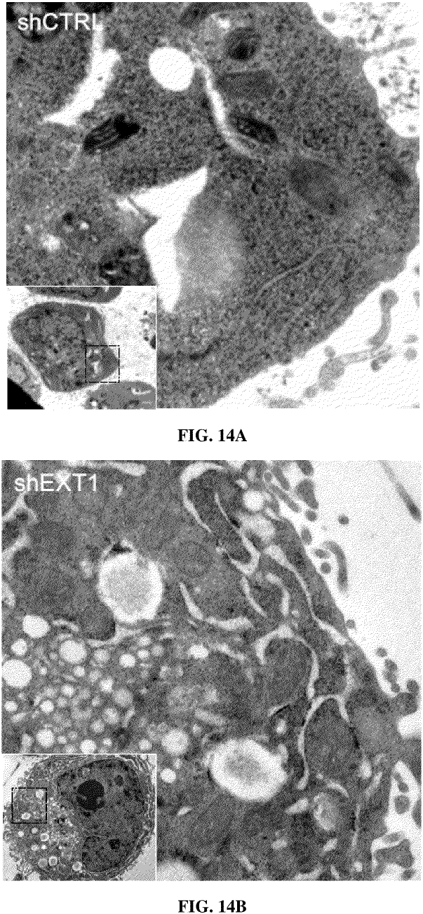

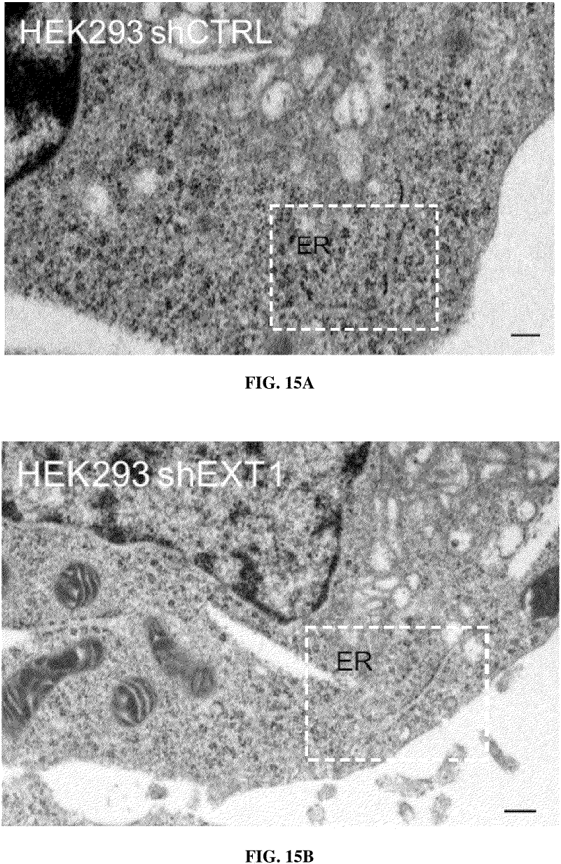

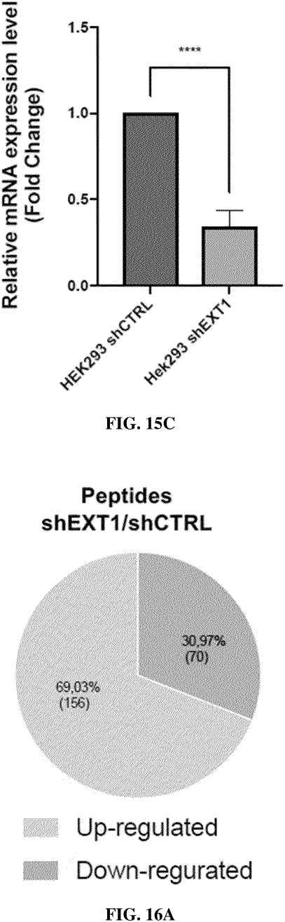

In the Golgi apparatus, EXT1 catalyzes the polymerization of HS chain. TEM ultrastructural examination of KD _EXT1 cells revealed structural changes in the Golgi apparatus size and shape (FIGS. 10A-B). The number of Golgi cisternae per stack was reduced from 3.80±0.98 to 3.00±0.86 (shCTRL and shEXT1, respectively) (FIGS. 11A-B, FIG. 12) and stacks appeared dilated and upon quantification showed shorter length (1,036±312 nm compared to 729.2±329.0 nm, shCTRL and shEXT1, respectively) in KD_EXT1 compared to control cells (FIGS. 11A-B, FIG. 13). The ultrastructural ER morphology was subsequently assessed and it was observed well-organized ER tubular extensions in HeLa KD_EXT1 cells, with an average length of 109.60±25.29 µm compared to 19.00±8.02 µm in control cells (FIGS. 14A-B). These observations are in agreement with the above results demonstrating a perturbation of ER-to-Golgi, and trans-Golgi secretory vesicles system and support coordinated biogenesis and maintenance of ER and Golgi structures. Similar ER morphology defects were also observed in other cell types, including HEK293 (FIGS. 15A-C), Jurkat, and ex-vivo activated T-cells from peripheral lymph organs. Also, depletion of other members of the exostosin family (EXT2, EXTL1-3) did not lead to similar ER defects.

### 2.5- Reprogramming the Proteome and the Glycome, in the ER Membranes

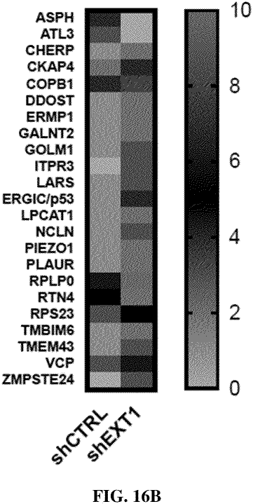

To understand the molecular mechanism of EXT 1-mediated ER membrane structuration, ER microsomes were isolated from KD_EXT1 and control cells. TEM revealed that ER membrane fragments of KD_EXT1 cells appeared vesicle-like, compared to the normal heterogeneous microsomes observed in control cells. Compared to control, microsomes isolated from KD_EXT1 cells were depleted in various ER-resident proteins, including the luminal chaperone calnexin, the ER-integrated components of the translocon complex Sec62 and Sec63, the translocon-associated protein complex (TRAP) and the oligosaccharyl-transferase complex (OST) members STT3A, STT3B and Dad1, further confirming the involvement of EXT1 in protein transport and targeting to the ER membranes. To evaluate the global role of EXT1 in the ER membrane composition, the proteome, lipidome, and glycome of ER membrane were comprehensively profiled from control and KD_EXT1 cells. 226 proteins differentially expressed in ER membranes depleted for EXT1 were identified, including 23 ER-resident proteins (FIGS. 16A-B). While RTN4 and ATL3 shaping proteins were downregulated, proteins such as valosin-containing protein (VCP), an ATPase involved in lipids recruitment during transitional ER formation, and glycan-binding protein ERGIC/p53, a component of the ER-Golgi intermediate compartment involved in ER reorganization for cargo transport, were up-regulated in KD_EXT1 ER membranes (FIG. 16B), further confirming the above observations on secretion.

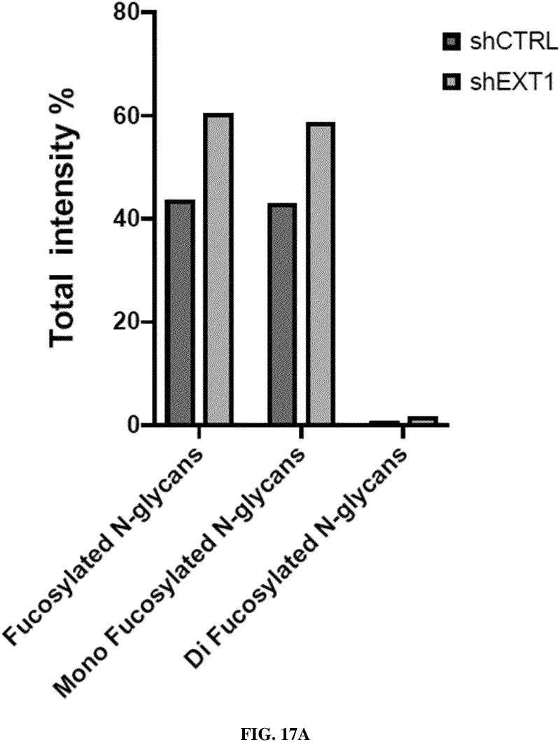

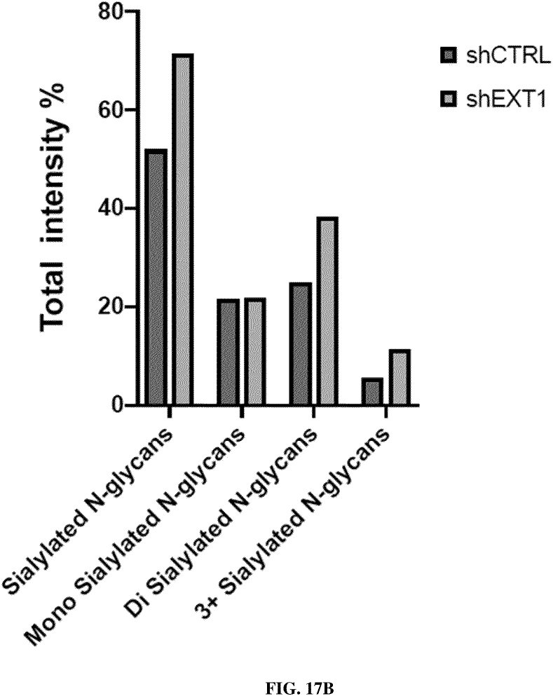

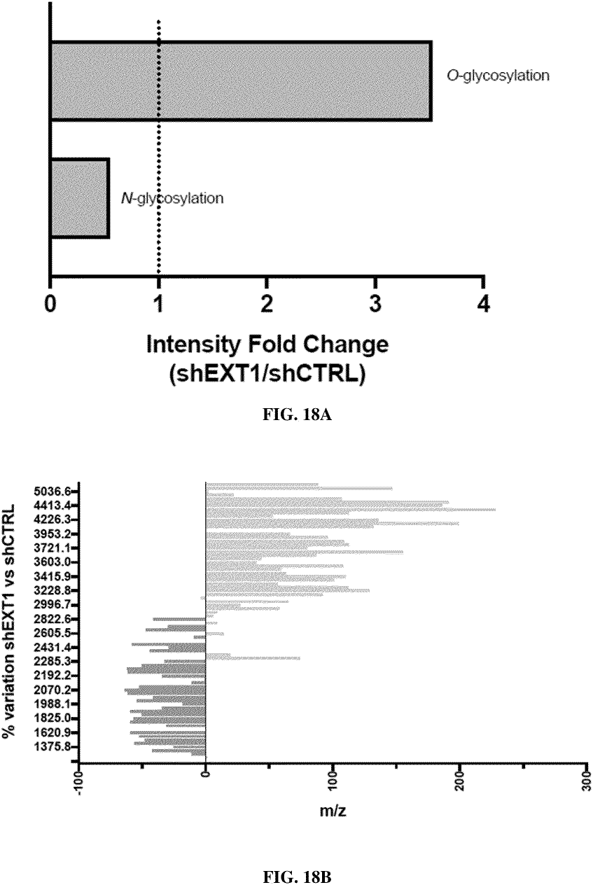

N- and O-glycans were next quantified by MALDI-TOF-MS, enabling absolute and relative estimation of glycans abundance on glycoproteins. Knockdown of EXT1 did not change the composition of glycans on membrane proteins (FIGS. 17A-B). However, the total amount of N-glycans was reduced, and we observed a significant shift towards higher molecular weight glycans compared to control ER membranes (FIGS. 18A-B). This deregulation appears to occur at the level of the first step during protein N-glycosylation involving the OST complex, whose catalytic subunits STT3A and STT3B are reduced following EXT1 depletion. The specific N-glycans attached to asparagine (N) residues of STT3A, STT3B and RPN1 OST subunits were identified. The corresponding sequon of yeast Stt3 was shown, by cryo-EM, to mediate the assembly of the OST subcomplexes via interaction with Wbp1 and Swp1. Depletion of EXT1 induced less N-glycosylation of the OST catalytic subunits (STT3A and STT3B) at N548 and N627 residues, respectively, confirming the observation that EXT1 is involved in the stability of the OST complex in ER microsomes. It was also observed an increase in O-glycans in KD_EXT1 ER membranes (FIG. 18A), consistent with their higher content in GalNAc transferase 2 (GALNT2) and the overall higher glycosyltransferase activity in ER microsomes following knockdown of EXT1. These results indicate that depletion of EXT1 leads to a displacement of glycosylation equilibrium of ER membrane proteins.

### 2.6- Biological Significance

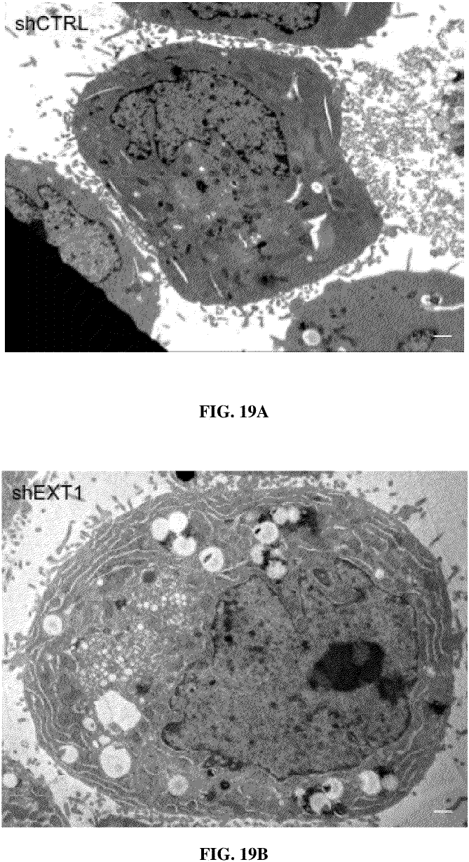

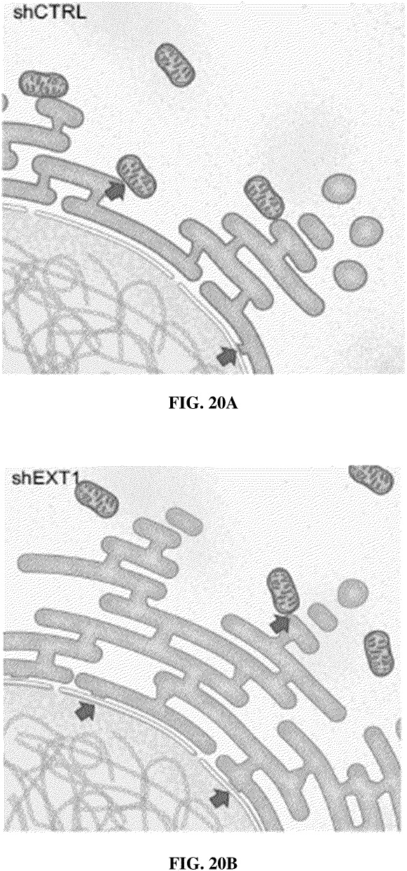

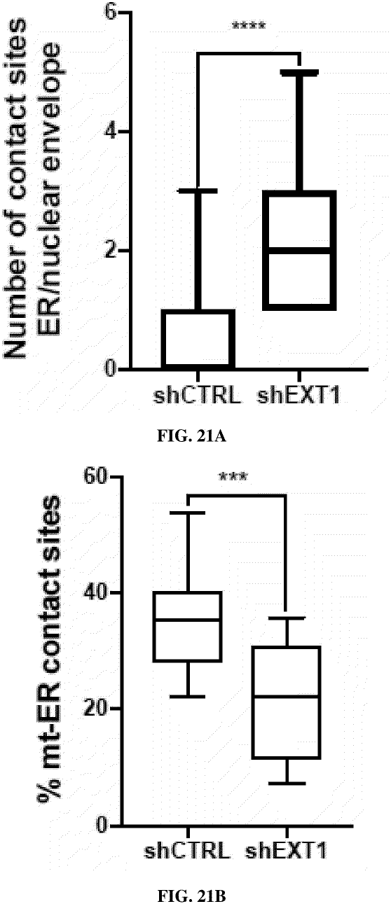

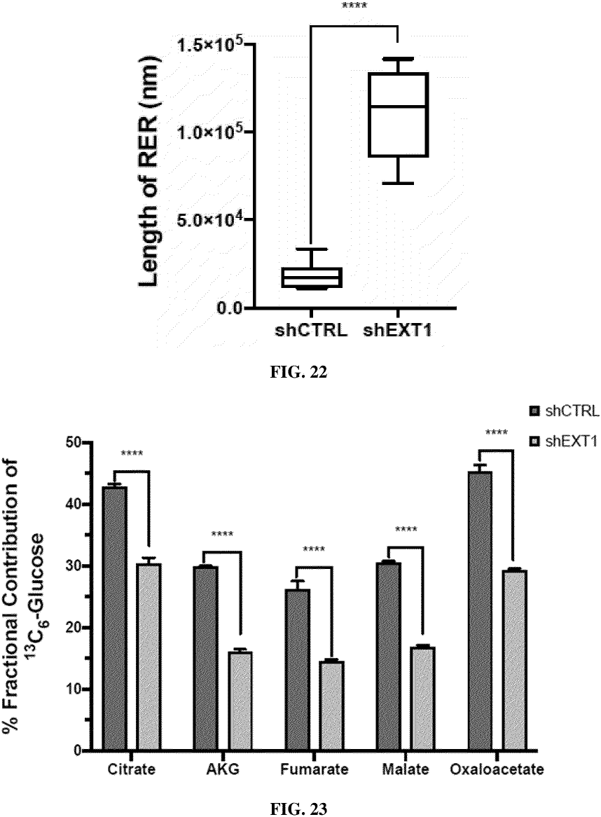

The Hela cellsize was examined and it was observed that ER extension in KD _EXT1 cells correlated with a ~2-fold increase in cellular area (68.52±12.52 and 133.9±36.79 µm² in shCTRL and shEXT1, respectively) (FIGS. 19A-B). Cell size is of fundamental importance from bacteria to mammals, and it is strictly regulated to keep a balance between cell growth and cell division. Interestingly, it was not observed any significant effect on proliferation following EXT1 depletion suggesting an important adaptive change of the size threshold following ER extension and internal cellular architecture rearrangement in KD_EXT1 cells. ER interactions with other organelles were next analyzed and it was possible to count significantly more peripheral ER-nuclear envelope (2.30±1.18 and 0.55±0.85 shEXT1 and shCTRL, respectively) and less ER-mitochondria (21.6%±10.2 and 35.38%±9.32 shEXT1 and shCTRL, respectively), contact sites in KD_EXT1 compared to control condition (FIGS. 20A-B, FIGS. 21A-B). The latter observation was highly unexpected given the ~5,7-fold increase in ER length (FIG. 22). However, it correlated with an impaired calcium flux and loss of interaction between EXT1 and the Sarco/endoplasmic reticulum Ca²⁺ ATPase 2. Taken together, the above results suggest that cells underwent a metabolic switch following EXT1 knockdown.

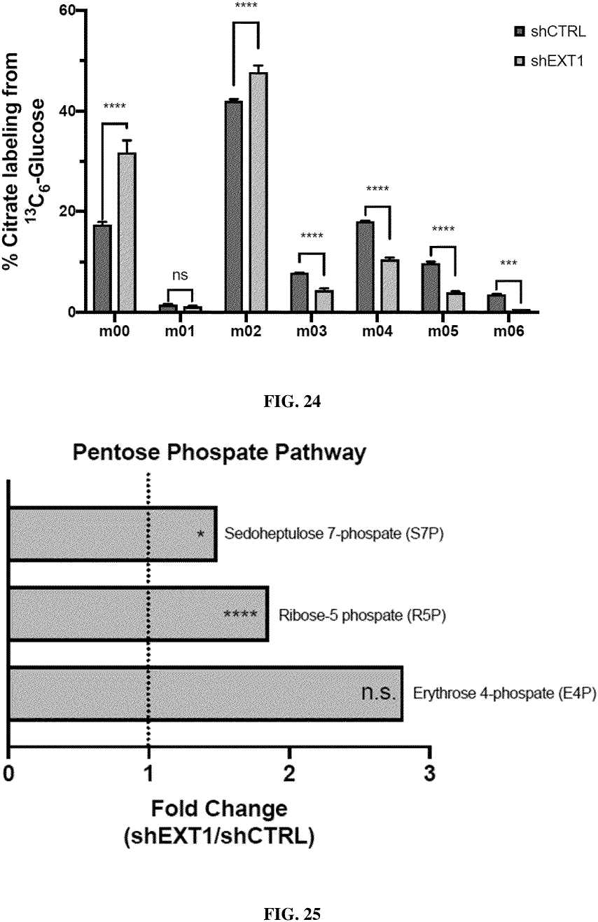

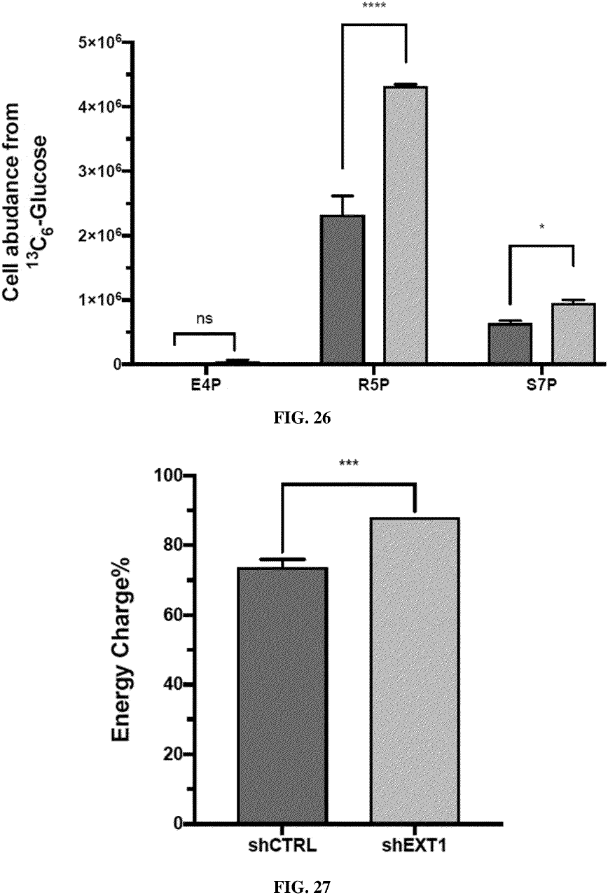

To further assess the implications of EXT1 in cell metabolism, two different strategies were used. Firstly, based on previous transcriptomics data in cells treated with siRNA targeting EXT1 and control cells (Daakour et al.; see above), two in silico flux balance analysis (FBA) models were reconstructed using Constraint-Based Reconstruction Analysis (COBRA) tools based on human RECON2 metabolic model. 34 and 39 reactions were uniquely found active in the KD_EXT1 or control models, respectively. These reactions are involved in the Tricarboxylic Acid (TCA) cycle, glycerophospholipid metabolism, pyruvate, methane, and sphingolipid metabolism. These predictions were confirmed with high throughput metabolomic analysis of the relative abundance and fractional contribution of intracellular metabolites from major metabolic pathways in living cells. It was not observed significant changes in glycolysis between control and KD_EXT1 cells. In contrast, it was found that several nucleotides, amino acids, and metabolites from the TCA cycle were dysregulated in cells depleted for EXT1, in agreement with our FBA in-silico analysis. The fractional contribution of glucose carbons into these pools of metabolites was also decreased in KD_EXT1 cells (FIG. 23). For instance, citric acid (change 12.51%, p < 0.001), a-ketoglutarate (change 13.87%, p < 0.0001), fumarate (change 11.61%, p < 0.001), malate (change 13.74%, p < 0.0001) and oxaloacetate (change 15.97%, p < 0.0001) showed significant drops in fractional contribution (FIG. 23). Iso-topologue profile analysis of TCA intermediates pointed towards a less oxidative mode of action of the mitochondria of cells depleted for EXT1, as evidenced by the drop in iso-topologues m04, m05 and m06 of citric acid (FIG. 24). In contrast, metabolite pools of the pentose phosphate pathway, as well as the m05 of different nucleotides (ATP, UTP, GTP, and CTP), and the energy charge was increased in the KD_EXT1 cells (FIGS. 25-27), indicating a higher de novo synthesis and consumption rate of these nucleotides necessary for the synthesis of sugar intermediates such as UDP-hexoses and UDP-GlcNAc.

### 2.7- Discussion

In vitro, the formation of the ER tubular network requires only a small set of membrane-curvature and stabilizing proteins that includes RTNs, REEPs, and large ATLs GTPases. However, these effectors cannot account for the diversity and adaptability of ER size and morphology observed in individual cell types. It is expected that in vivo, dynamics of tubular three-way junctions and rearrangements of the tubules to accommodate luminal flow mobility rely on additional proteins or mechanisms. Despite the discovery of glycoproteins in intracellular compartments 30 years ago, the knowledge about the glycoproteome is still biased towards secreted and plasma membrane proteins, including cell surface receptors and peripheral membrane proteins, for which glycosylation heavily influences their function. Here, it was demonstrated that, by depleting a single ER-resident glycosyltransferase, EXT1, we could induce an alternative glycosylation pattern of ER membrane proteins and lipids that correlates with extensive ER architectural and functional remodeling.

These findings suggest an adaptive cellular mechanism that facilitates the equilibrium towards complex N-glycosylation and redistribution of HSPGs when EXT1 is depleted.

Herein is provided a new edge to the role of EXT1 in cell physiology, besides heparan sulfate biosynthesis at the cell surface. EXT1 is required for dictating macromolecules composition that govern ER morphology and luminal trafficking. At the fundamental level, these findings argue for a general biophysical model of ER membrane-extension and functions regulated by resident glycosyltransferase enzymes.

### Example 2: Depletion of EXT1 in HEK293T and in HeLa Cell Lines Increases The Production of Recombinant Proteins and Viral Particles

### 1- Materials and Methods

### 1.1- Lentiviral Production

HEK293 cells (shEXT1 or ShCTRL) are cultivated in 175 cm² bottle at a density of 10⁶ cells and incubated at 37° C. with 5% CO₂ for 72 hours in DMEM (Dulbecco’s Modified Eagle Medium) with 10% FBS. Prior to transfection, the media is changed and cells are co-transfected with the packaging plasmid psPAX2, envelop plasmid pVSV-G and the transfer plasmid coding for EmGFP, using the calcium-phosphate method. Cells are left in the incubator for 24 hours, the media is changed and replaced by 12 ml of fresh DMEM for additional 24 hours. The supernatant is treated with DNase for 20 min at 37° C., filtered under 0.20 µm, centrifuged for 1h45 min at 16,000 rpm and the viral pellets were suspended into 300 µl of HEPES buffer. Virus titration is performed by qPCR using the LV900 kit (www.abmgood.com).

### 1.2- Adeno Associated Virus Production

HEK293 cells (shEXT1 or ShCTRL) are cultivated in 175 cm² bottle at a density of 1,7× 10⁶ cells and incubated at 37° C. with 5% CO₂ for 72 hours in DMEM with 10% FBS. Prior to transfection, the media is changed and cells are co-transfected with plasmids RepCap, pHelper and the transfer plasmid coding for the red fluorescent protein (RFP), using the calcium-phosphate method. Cells are left in the incubator for 12 hours; the media is changed and replaced by fresh DMEM for additional 72 hours. Cells are harvested with media and centrifuged at 1,000xg for 10 min at 4° C. Viruses in the 100 ml of supernatant are obtained by incubating with 25 ml of 40% PEG, followed by a centrifugation of the precipitated viruses at 3,000xg for 15 min at 4° C. Viruses in the cell pellets are obtained after cells lysis by 3 cycles of freeze-thaw, centrifugation at 3,000xg for 15 min at 4° C. The protocol for viruses purification and validation is detailed at https://www.addgene.org/protocols/aav-purification-iodixanol-gradient-ultracentrifugation/. Virus titration is performed by qPCR using the ABMGood G931 kit (www.abmgood.com).

### 1.3- Protein Production

HEK293 cells (shEXT1 or ShCTRL) are cultivated in 175 cm² bottle at a density of 10⁶ cells and incubated at 37° C. with 5% CO₂ for 72 hours in DMEM with 10% FBS. Prior to transfection, the media is changed and cells are transfected with a plasmid expressing Notch1 tagged with Flag epitope. Cells are left in the incubator for 12 hours; the media is changed and replaced by fresh DMEM for additional 72 hours. Cells are harvested with media and centrifuged at 1000×g for 10 min at 4° C. Cells are lysed with 1% Tween and analyzed by western blot using an anti-Flag antibody.

In another experiment, HEK293 cells (shEXT1 or ShCTRL) are infected with VSVG lentiviruses with a transfer plasmid coding for the nano-luciferase enzyme. Cells are left in the incubator for 24 hours; and the nano-luciferase is measured using nano-Glo luciferase assay system (www.promega.com)

### 2. Results

### 2.1- Lentiviral Production

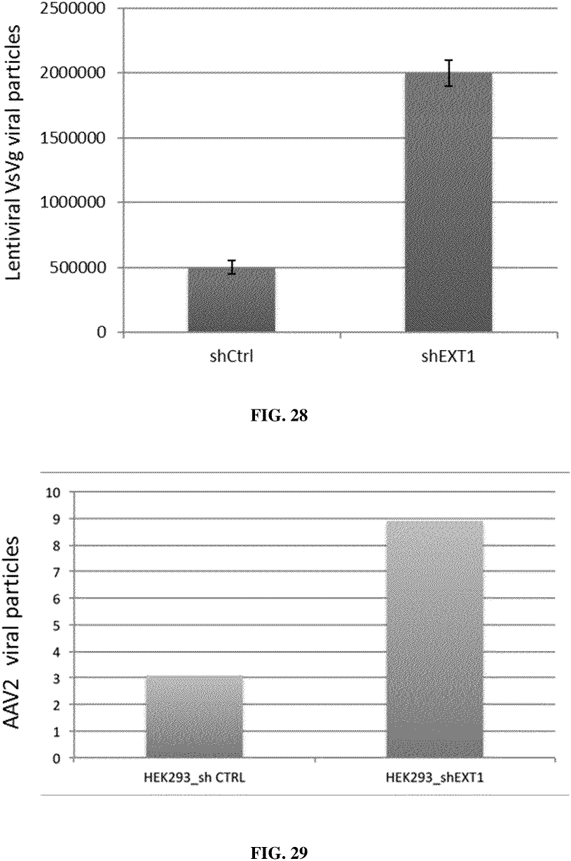

HEK293 shEXT 1 produce approximately four times more viruses than HEK293 shCTRL cells (respectively 2×10⁶ versus 5×10⁵ lentiviral particles/ml) (FIG. 28).

### 2.2- Adeno Associated Virus Production

HEK293 shEXT1 produce approximately three times more AAV2 pseudo-typed viruses than HEK293 shCTRL cells (respectively 8.9×10¹² vs 2.8×10¹² viral particles/ml) (FIG. 29).

### 2.3- Protein Production

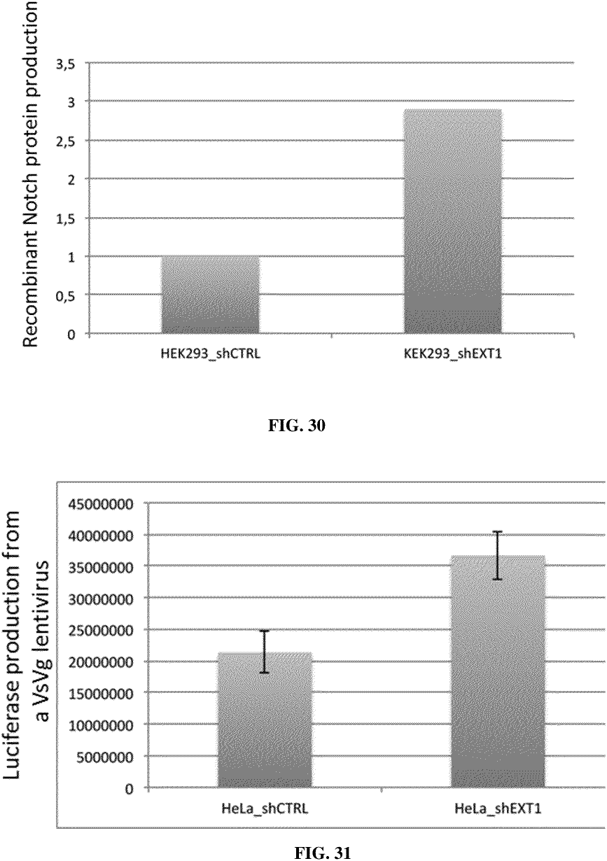

HEKshEXT1 cells express 2.9 times more Notchl-Flag protein than control cells (FIG. 30). HEKshEXTl cells express 1.7 times more nano-luciferase enzyme than control cells (FIG. 31).

### Example 3: siRNA Efficiently Deplete Cells of EXT1

In addition to shRNA, two different siRNA were used to demonstrate that EXT1 knockdown affect the protein secretory pathway. To this end Calnexin marker was used, which is a protein involved in quality control of the secretory pathway. Cells treated with shRNA or siRNA EXT1 were stained with a mouse antibody for calnexin (www.abcam.com). Accordingly, the molecular chaperone calnexin, which assists protein folding in the ER, exhibited an aggregation pattern in KD_EXT1 cells. This aggregation might result in a decreased movement of molecules through the ER lumen.

### Example 4: Additional shRNAs Efficiently Deplete Cells of EXT1

### 1. Methods

shRNA (Table 4) targeting human EXT1 gene and, as a control, an irrelevant sequence (shRNA control) were cloned into a lentiviral plasmid containing an ampicillin and puromycin resistant genes for selection in bacteria and in animal cells respectively. The plasmids were amplified using E. coli DH5 strain (Thermo Fisher Scientific®, Cat# 18265017), and DNA midi-preparation performed using a NucleoBond Xtra midi kit from Macherey-Nagel® (Cat# REF 740410.50).

TABLE 4

selected nucleic acids encoding shRNA sequences targeting human EXT1

| | | |
|---|---|---|
| Vector Name | EXT1 mRNA Target sequence | SEQ ID NO: |
| pLV[shRNA]-Puro-U6>hEXT1 [shRNA#1] | ATTCTTGTGGGAGGCTTATTT | SEQ ID NO: 33 |
| pLV[shRNA]-Puro-U6>hEXT1 [shRNA#2] | CCTTCTACAATCAGGTCTATT | SEQ ID NO: 34 |
| pLV[shRNA]-Puro-U6>hEXT1 [shRNA#3] | CCCAACTTTGATGTTTCTATT | SEQ ID NO: 35 |
| pLV[shRNA]-Puro-U6>hEXT1 [shRNA#4] | GAGTATGAGAAGTATGATTAT | SEQ ID NO: 36 |
| pLV[shRNA]-Puro-U6>hEXT1 [shRNA#5] | CTTCGTTCCTTGGGATCAATT | SEQ ID NO: 37 |
| pLV[shRNA]-Puro-U6>hEXT1 [shRNA#6] | AGCCAGATTGTGCCAACTATC | SEQ ID NO: 38 |
| pLV[shRNA]-Puro-U6>hEXT1 [shRNA#7] | ATTTCGGAGGCTTGCAGTTTA | SEQ ID NO: 39 |
| pLV[shRNA]-Puro-U6>hEXT1 [shRNA#8] | GTCCTGAGTCTGGATACTTTA | SEQ ID NO: 40 |
| pLV[shRNA]-Puro-U6>hEXT1 [shRNA#9] | GCACTTAGACAGCAGACACAA | SEQ ID NO: 41 |
| pLV[shRNA]-Puro-U6>hEXT1 [shRNA#10] | GAAGAACACAGCGGTAGGAAT | SEQ ID NO: 42 |
| pLV[shRNA]-Puro-U6>hEXT1 [shRNA#11] | CAATTGTGAGGACATTCTCAT | SEQ ID NO: 43 |
| pLV[shRNA]-Puro-U6>hEXT1 [shRNA#12] | CCTGCTTCGATTTCACCCTTT | SEQ ID NO: 44 |
| pLV[shRNA]-Puro-U6>hEXT1 [shRNA#13] | CCTCAGTATGTGCACAATTTG | SEQ ID NO: 45 |
| pLV[shRNA]-Puro-U6>hEXT1 [shRNA#14] | AGACACCAGGAATGCCTTATA | SEQ ID NO: 46 |
| pLV[shRNA]-Puro-U6>hEXT1 [shRNA#15] | TGCCATTCTCTGAAGTGATTA | SEQ ID NO: 47 |
| pLV[shRNA]-Puro-U6>hEXT1 [shRNA#16] | GGCGATGAGAGATTGTTATTA | SEQ ID NO: 48 |
| pLV[shRNA]-Puro-U6>hEXT1 [shRNA#17] | CAGTTGAGAAGATTGTATTAA | SEQ ID NO: 49 |
| pLV[shRNA]-Puro-U6>hEXT1 [shRNA#18] | CAATGGTAGGAATCATTTAAT | SEQ ID NO: 50 |
| pLV[shRNA]-Puro-U6>hEXT1 [shRNA#19] | TCCTTACTACTATGCTAATTT | SEQ ID NO: 51 |
| pLV[shRNA]-Puro-U6>hEXT1 [shRNA#20] | GTTGACAGGAGCTGCTATTTA | SEQ ID NO: 52 |
| pLV[shRNA]-Puro-U6>hEXT1 [shCTRL] | TCCGCAGGTATGCACGCGTGAATT | SEQ ID NO: 53 |

10×10⁶ HEK293 cells (ATCC®# CRL 1573) were cultured in Dulbecco’s Modified Eagle Medium (DMEM) supplemented with 10% fetal bovine serum, 2 mmol/L L-glutamine and 100 I.U./ml penicillin and 100 µg/ml streptomycin. Cells were incubated at 37° C. with 5% CO₂ and 95% humidity. Cells we transfected with 10 µgof each DNA construct (Table 4) using 10 µl of Polyethylenimine (MW 25,000, Polysciences® cat# 9002-98-6). Forty-eight hours post-transfection, cells were cultured in the presence of 1 µg/ml of puromycin (Sigma Aldrich®, Cat# P8833), to select for the expression of shRNA molecules. After selection, resistant cells were amplified and frozen.

For western blot, cells were lysed in immunoprecipitation low salt buffer (IPLS: 25 mM Tris-HCl pH 7.4, 150 mM NaCl, 1 mM EDTA, 1% NP-40, and 5% glycerol, complete Protease Inhibitor (Roche®) and Halt Phosphatase Inhibitors (Thermo Fisher Scientific®)). SDS-PAGE and western blotting were performed using standard protocols. The following primary antibodies were used: rabbit-anti-GAPDH 1:2,000 (Abeam® ab8245), rabbit-anti-EXT1 1:500 (Prestige Antibodies, Sigma-Aldrich®, cat# HPA044394). A secondary anti-rabbit HRP-conjugated antibody (Santa Cruz®, Cat# sc-2357) was finally used to reveal positive immunoblotting.

For viral infectivity and transgene expression, the different EXT1-targeting shRNAs (#1 to #20) expressing HEK293 cell lines were cultured in 24-well plates in DMEM supplemented with 10% fetal bovine serum, 2 mmol/1 L-glutamine and 100 I.U./ml penicillin and 100 µg/ml streptomycin. Cells were then infected with lentiviral or AAV2 particles expressing Nano-luciferase (NLuc) enzyme or green fluorescent protein (GFP), respectively. Twenty-four hours post-infection, NLuc activities or GFP fluorescence intensities were quantified using a Nanoluciferase kit (Promega® cat# N1120), or the Incucyte S3 live cells instrument (Sartorius®).

### 2. Results

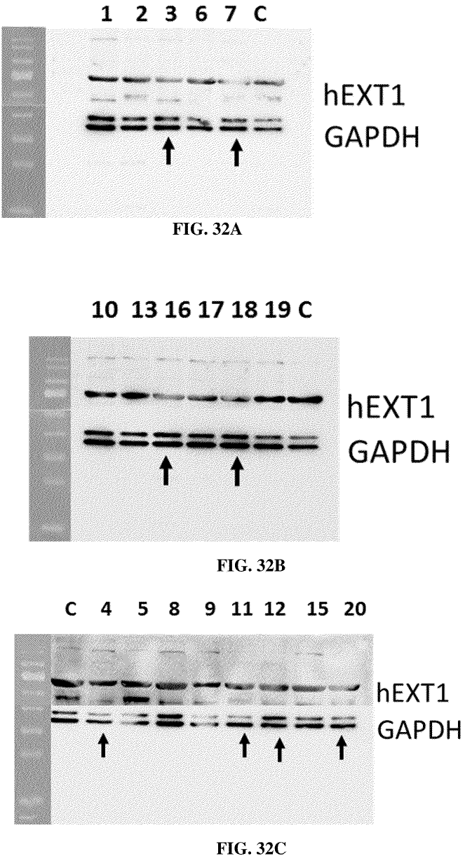

Examination of the western blot results, after immunoblotting of cell lysates using anti-EXT1 and anti-GAPDH (FIGS. 32A-C), indicates that not all shRNA sequences used are able to reduce the levels of EXT1 expression in HEK293 cells. We identified 8 out of 20 tested shRNA sequences able to induce reduction of EXT1 levels in cells (Table 5).

TABLE 5

nucleic acids encoding selected shRNA sequences targeting human EXT1

| | | |
|---|---|---|
| shRNA# | EXT1 mRNA Target sequence | SEQ ID NO: |
| shRNA#3 | CCCAACTTTGATGTTTCTATT | SEQ ID NO: 35 |
| shRNA#4 | GAGTATGAGAAGTATGATTAT | SEQ ID NO: 36 |
| shRNA#7 | ATTTCGGAGGCTTGCAGTTTA | SEQ ID NO: 39 |
| shRNA#11 | CAATTGTGAGGACATTCTCAT | SEQ ID NO: 43 |
| shRNA#12 | CCTGCTTCGATTTCACCCTTT | SEQ ID NO: 44 |
| shRNA#16 | GGCGATGAGAGATTGTTATTA | SEQ ID NO: 48 |
| shRNA#18 | CAATGGTAGGAATCATTTAAT | SEQ ID NO: 50 |
| shRNA#20 | GTTGACAGGAGCTGCTATTTA | SEQ ID NO: 52 |

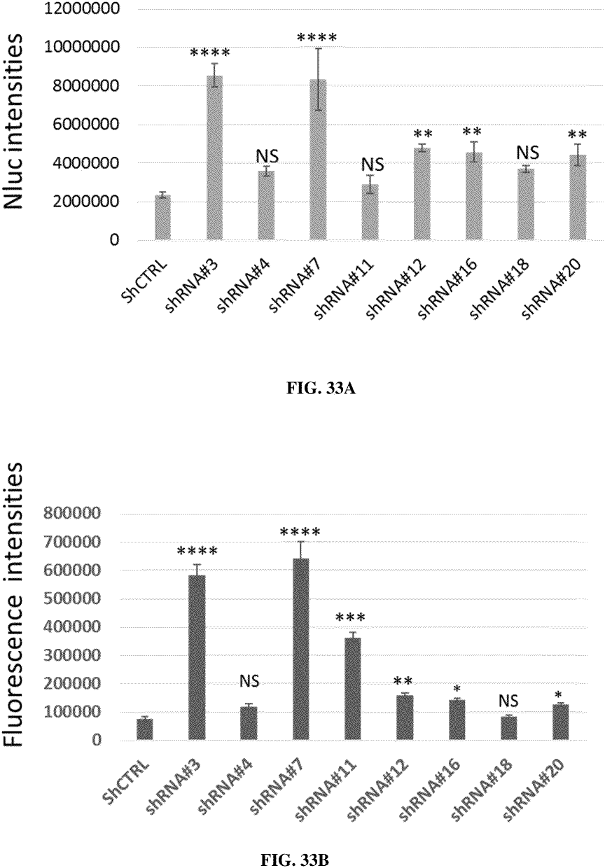

To examine whether HEK293 knocked down for EXT1 expression could express transgenes from lentiviral particles and AAV2 serotypes viruses, we transduced knockdown confirmed cells with a nanoluciferase expressing lentivirus or a GFP expressing AAV2 virus at 1 plaque-forming unit (PFU). shRNA#3 and # 7 exhibited the highest productivity in both lentiviral and AAV systems (FIGS. 33A-B). Other EXT1 knockdown cells lines also showed significant productivity compared to controls cells, namely shEXT1#12, 16 and 20 for lentiviruses (FIG. 33A); and shEXT1# 11, 12, 16 and 20 for AAV viruses (FIG. 33B).

### 3. Conclusion

In addition to characterized shRNA and siRNA sequences targeting EXT1 (see examples 1-3), 8 additional sequences targeting EXT1 were further validated (Table 5), and a positive correlation between knockdown of EXT1 and HEK293 cell productivity was hereby confirmed.

1-15. (canceled)

16. A method for the production of a biological entity in a cell, said method comprising the steps of:
a) providing a cell population having at least depleted EXT1 expression and/or activity;
b) transfecting the cell population of step a) with an oligonucleotide encoding the biological entity, preferably a polypeptide or a viral particle.

17. The method according to claim 16, wherein said cell is a eukaryotic cell.

18. The method according to claim 16, wherein said biological entity is selected in a group comprising a recombinant polypeptide and/or a viral particle.

19. The method according to claim 16, wherein said cell comprises a partial or total knockout of the EXT1 gene.

20. The method according to claim 16, wherein said at least depleted EXT1 expression and/or activity is obtained by the treatment of said cell with an inhibitor of EXT1 expression and/or activity.

21. The method according to claim 20, wherein said inhibitor of the EXT1 expression and/or activity is selected from a group comprising an oligonucleotide, an aptamer, an oligopeptide, a polypeptide, a chemical compound and an analog thereof.

22. The method according to claim 20, wherein said inhibitor of the EXT1 expression is selected from a group comprising or consisting of an antisense RNA, a miRNA, a guide RNA, a siRNA, and a shRNA.

23. The method according to claim 20, wherein said inhibitor of the EXT1 expression and/or activity is selected in a group comprising an oligonucleotide having at least 75% identity with any one of sequences SEQ ID NO: 1 to SEQ ID NO: 27 and SEQ ID NO: 33 to SEQ ID NO: 53.

24. The method according to claim 20, wherein said inhibitor of the EXT1 expression and/or activity is selected in a group comprising an oligonucleotide represented by any one of sequences SEQ ID NO: 1 to SEQ ID NO: 27 and SEQ ID NO: 33 to SEQ ID NO: 53.

25. The method according to claim 16, wherein said method further comprises the step of:
d) extracting and/or purifying the synthesized polypeptide or viral particle.

26. A method for the production of a biological entity in a cell, said method comprising the steps of:
a) providing a cell population;
b) transfecting the cell population of step a) with an oligonucleotide encoding the biological entity.
c) inhibiting EXT1 expression in the said cell by using an inhibitor of EXT1 expression and/or activity.

27. The method according to claim 26, wherein said cell is a eukaryotic cell.

28. The method according to claim 26, wherein said biological entity is selected in a group comprising a recombinant polypeptide and/or a viral particle.

29. The method according to claim 26, wherein said inhibitor of the EXT1 expression and/or activity is selected from a group comprising an oligonucleotide, an aptamer, an oligopeptide, a polypeptide, a chemical compound and an analog thereof.

30. The method according to claim 26, wherein said inhibitor of the EXT1 expression is selected from a group comprising or consisting of an antisense RNA, a miRNA, a guide RNA, a siRNA, and a shRNA.

31. The method according to claim 26, wherein said inhibitor of EXT1 expression and/or activity is an oligonucleotide having at least 75% identity with any one of sequences SEQ ID NO: 1 to SEQ ID NO: 27 and SEQ ID NO: 33 to SEQ ID NO: 53.

32. The method according to claim 26, wherein said inhibitor of the EXT1 expression and/or activity is an oligonucleotide represented by any one of sequences SEQ ID NO: 1 to SEQ ID NO: 27 and SEQ ID NO: 33 to SEQ ID NO: 53.

33. The method according to claim 26, wherein said method further comprises the step of:
d) culturing the transfected cell population obtained at step b) in a suitable culture medium, so as to synthesize the polypeptide or the viral particle.

34. The method according to claim 33, wherein said method further comprises the step of:
e) extracting and/or purifying the synthesized polypeptide or viral particle.


SEQUENCE LISTING (53 sequences)

SEQ ID NO: 1 - DNA, 58 bp, Artificial Sequence
  shEXT1-1
ccggagagccagattgtgccaactactcgagtagttggcacaatctggctcttttttg

SEQ ID NO: 2 - DNA, 58 bp, Artificial Sequence
  shEXT1-2
ccggccttcgttccttgggatcaatctcgagattgatcccaaggaacgaaggtttttg

SEQ ID NO: 3 - DNA, 41 bp, Artificial Sequence
  shEXT1-3
agcagacacaattcttgtgggaggcttatttttcttcagtt

SEQ ID NO: 4 - DNA, 41 bp, Artificial Sequence
  shEXT1-4
attacagattccttctacaatcaggtctattcatcaggata

SEQ ID NO: 5 - DNA, 41 bp, Artificial Sequence
  shEXT1-5
aaacttccgacccaactttgatgtttctattcccctctttt

SEQ ID NO: 6 - DNA, 41 bp, Artificial Sequence
  shEXT1-6
agacaacaccgagtatgagaagtatgattatcgggaaatgc

SEQ ID NO: 7 - DNA, 41 bp, Artificial Sequence
  shEXT1-7
ctctgcgccccttcgttccttgggatcaattggaaaacgag

SEQ ID NO: 8 - DNA, 41 bp, Artificial Sequence
  shEXT1-8
tcatcagcagagccagattgtgccaactatccaaaaactta

SEQ ID NO: 9 - DNA, 41 bp, Artificial Sequence
  shEXT1-9
gctctgcgccccttcgttccttgggatcaattggaaaacga

SEQ ID NO: 10 - DNA, 41 bp, Artificial Sequence
  shEXT1-10
actcatcagcagagccagattgtgccaactatccaaaaact

SEQ ID NO: 11 - DNA, 41 bp, Artificial Sequence
  shEXT1-11
taaaatcctagcacttagacagcagacacaattcttgtggg

SEQ ID NO: 12 - DNA, 41 bp, Artificial Sequence
  shEXT1-12
cagccggagagaagaacacagcggtaggaatggcttgcacc

SEQ ID NO: 13 - DNA, 41 bp, Artificial Sequence
  shEXT1-13
accaattggccaattgtgaggacattctcatgaacttcctg

SEQ ID NO: 14 - DNA, 41 bp, Artificial Sequence
  shEXT1-14
cgcatggagtcctgcttcgatttcaccctttgcaagaaaaa

SEQ ID NO: 15 - DNA, 58 bp, Artificial Sequence
  shEXT1-15
ccggcccaactttgatgtttctattctcgagaatagaaacatcaaagttgggtttttg

SEQ ID NO: 16 - DNA, 58 bp, Artificial Sequence
  shEXT1-17
ccgggcacttagacagcagacacaactcgagttgtgtctgctgtctaagtgctttttg

SEQ ID NO: 17 - DNA, 58 bp, Artificial Sequence
  shEXT1-18
ccggcctgcttcgatttcaccctttctcgagaaagggtgaaatcgaagcaggtttttg

SEQ ID NO: 18 - DNA, 58 bp, Artificial Sequence
  shEXT1-20
ccggcaagactaggttggtacagttctcgagaactgtaccaacctagtcttgtttttg

SEQ ID NO: 19 - DNA, 57 bp, Artificial Sequence
  shEXT1-21
ccggaagaacacagcggtaggaatctcgagattcctaccgctgtgttcttctttttg

SEQ ID NO: 20 - DNA, 58 bp, Artificial Sequence
  shEXT1-22
ccggcaattgtgaggacattctcatctcgagatgagaatgtcctcacaattgtttttg

SEQ ID NO: 21 - DNA, 58 bp, Artificial Sequence
  shEXT1-23
ccggattcttgtgggaggcttatttctcgagaaataagcctcccacaagaattttttg

SEQ ID NO: 22 - DNA, 58 bp, Artificial Sequence
  shEXT1-24
ccggagccagattgtgccaactatcctcgaggatagttggcacaatctggcttttttg

SEQ ID NO: 23 - DNA, 58 bp, Artificial Sequence
  shEXT1-25
ccggcttcgttccttgggatcaattctcgagaattgatcccaaggaacgaagtttttg

SEQ ID NO: 24 - DNA, 58 bp, Artificial Sequence
  shEXT1-27
ccgggagtatgagaagtatgattatctcgagataatcatacttctcatactctttttg

SEQ ID NO: 25 - RNA, 19 nt, Artificial Sequence
  siEXT1(1)
ggaucaucccaggacagga

SEQ ID NO: 26 - RNA, 19 nt, Artificial Sequence
  siEXT1(2)
ggauuccagcgugcacauu

SEQ ID NO: 27 - RNA, 19 nt, Artificial Sequence
  siEXT1(3)
ggcuuauuuuucuucaguu

SEQ ID NO: 28 - RNA, 19 nt, Artificial Sequence
  siCTRL
ggcugcuucuaugauuaug

SEQ ID NO: 29 - DNA, 21 bp, Artificial Sequence
  Primer EXT1 Forward
gctcttgtctcgcccttttgt

SEQ ID NO: 30 - DNA, 20 bp, Artificial Sequence
  Primer EXT1 Reverse
tggtgcaagccattcctacc

SEQ ID NO: 31 - DNA, 21 bp, Artificial Sequence
  Primer GAPDH Forward
ttgccatcaatgaccccttca

SEQ ID NO: 32 - DNA, 19 bp, Artificial Sequence
  Primer GAPDH Reverse
cgccccacttgattttgga

SEQ ID NO: 33 - DNA, 21 bp, Artificial Sequence
  shRNA#1
attcttgtgggaggcttattt

SEQ ID NO: 34 - DNA, 21 bp, Artificial Sequence
  shRNA#2
ccttctacaatcaggtctatt

SEQ ID NO: 35 - DNA, 21 bp, Artificial Sequence
  shRNA#3
cccaactttgatgtttctatt

SEQ ID NO: 36 - DNA, 21 bp, Artificial Sequence
  shRNA#4
gagtatgagaagtatgattat

SEQ ID NO: 37 - DNA, 21 bp, Artificial Sequence
  shRNA#5
cttcgttccttgggatcaatt

SEQ ID NO: 38 - DNA, 21 bp, Artificial Sequence
  shRNA#6
agccagattgtgccaactatc

SEQ ID NO: 39 - DNA, 21 bp, Artificial Sequence
  shRNA#7
atttcggaggcttgcagttta

SEQ ID NO: 40 - DNA, 21 bp, Artificial Sequence
  shRNA#8
gtcctgagtctggatacttta

SEQ ID NO: 41 - DNA, 21 bp, Artificial Sequence
  shRNA#9
gcacttagacagcagacacaa

SEQ ID NO: 42 - DNA, 21 bp, Artificial Sequence
  shRNA#10
gaagaacacagcggtaggaat

SEQ ID NO: 43 - DNA, 21 bp, Artificial Sequence
  shRNA#11
caattgtgaggacattctcat

SEQ ID NO: 44 - DNA, 21 bp, Artificial Sequence
  shRNA#12
cctgcttcgatttcacccttt

SEQ ID NO: 45 - DNA, 21 bp, Artificial Sequence
  shRNA#13
cctcagtatgtgcacaatttg

SEQ ID NO: 46 - DNA, 21 bp, Artificial Sequence
  shRNA#14
agacaccaggaatgccttata

SEQ ID NO: 47 - DNA, 21 bp, Artificial Sequence
  shRNA#15
tgccattctctgaagtgatta

SEQ ID NO: 48 - DNA, 21 bp, Artificial Sequence
  shRNA#16
ggcgatgagagattgttatta

SEQ ID NO: 49 - DNA, 21 bp, Artificial Sequence
  shRNA#17
cagttgagaagattgtattaa

SEQ ID NO: 50 - DNA, 21 bp, Artificial Sequence
  shRNA#18
caatggtaggaatcatttaat

SEQ ID NO: 51 - DNA, 21 bp, Artificial Sequence
  shRNA#19
tccttactactatgctaattt

SEQ ID NO: 52 - DNA, 21 bp, Artificial Sequence
  shRNA#20
gttgacaggagctgctattta

SEQ ID NO: 53 - DNA, 24 bp, Artificial Sequence
  shCTRL
tccgcaggtatgcacgcgtgaatt